(19) United States
(12) Patent Application Publication
(10) Pub. No.: US 2008/0213231 A1
(43) Pub. Date: Sep. 4, 2008

(54) Pluripotent Stem Cell Cloned From Single Cell Derived From Skeletal Muscle Tissue

(75) Inventors: Hidemasa Oh, Kyoto (JP); Tetsuya Nomura, Kyoto (JP); Hiroaki Matsubara, Kyoto (JP)

Correspondence Address: FITCH, EVEN, TABIN & FLANNERY, P. O. BOX 18415, WASHINGTON, DC, 20036, US

(73) Assignee: KYOTO UNIVERSITY, KYOTO (JP)

(21) Appl. No.: 11/988,836

(22) PCT Filed: Jul. 14, 2006
(86) PCT No.: PCT/JP2006/314070

§ 371 Date: Apr. 24, 2008

(30) Foreign Application Priority Data
Jul. 15, 2005 (JP) 2005-207670

(51) Int. Cl. A61K 35/12; C12N 5/06; C12Q 1/02; A61P 9/00

(52) U.S. Cl. 424/93.7; 435/266; 435/354; 435/381; 435/383; 435/29

(57) ABSTRACT

Techniques are provided which can isolate pluripotent stem cells at high purity capable of differentiation into at least a myocardial cell to regenerate the cardiac muscle. The pluripotent stem cells at high purity capable of differentiation into at least a myocardial cell to regenerate the cardiac muscle can be isolated through the following steps: (i) collecting a skeletal muscle tissue from a mammal and enzymatically treating the obtained skeletal muscle tissue to prepare a skeletal muscle tissue-derived cell; (ii) culturing the obtained skeletal muscle tissue-derived cell in a culture medium containing an epidermal growth factor and a fibroblast growth factor; (iii) selecting and separating a colony that is floating in the culture medium.

### TECHNICAL FIELD

The present invention relates to an isolated pluripotent stem cell derived from a skeletal muscle tissue and a method of isolating the pluripotent stem cell. The present invention also relates to a method of treating cardiac diseases by utilizing the pluripotent stem cell and a pharmaceutical composition comprising the same. Further, the present invention relates to a method of screening for a substance capable of differentiation induction or amplification of the pluripotent stem cell.

### BACKGROUND ART

Therapies for heart failures attributable to cardiac mechanical obstructions, myocardial insufficiency and rhythm abnormality, which have conventionally been carried out, involve symptomatic treatments such as reduction of blood flow by diuretics, enhancement of myocardial contractile force and proper regulation of pulsation of atrial flutter-fibrillation by cardiac stimulants, and reduction in cardiac load by vasodilators. For serious heart failures, on the other hand, no sufficient therapeutic effect can be obtained by the symptomatic treatments described above, and a basic remedy therefor by cardiac transplantation is required. However, cardiac transplantation has problems such as the shortage of donors, rejection reaction, and does not sufficiently function as relief healthcare at present. Accordingly, a method of transplanting progenitor cells or stem cells capable of differentiation into myocardial cells has attracted attention in recent years as relief healthcare as a substitute for cardiac transplantation.

However, previously reported cell transplantation can scarcely essentially regenerate myocardial cells, and a majority of cell transplantation techniques attempt to improve cardiac functions by a hematogenic improvement effect on microcirculation important for repair of ischemic myocardium or by a secondary myocardial protection effect of cytokines secreted from engrafted donor cells (see, for example, Patent Document 1).

Techniques of isolating stem cells capable of differentiation from mesenchymal stem cells or skeletal muscle cells into myocardial cells have been extensively examined (see, for example, Patent Document 2), but in these conventional techniques, the cells are purified with cell attachment or a specific cell surface antigen as the indicator, and are thus inevitably contaminated with cells other than the objective stem cells, thus resulting in a disadvantage that the purity of the isolated stem cells becomes extremely low. Such stem cells of low purity when used in cell transplantation may cause serious adverse effects and are thus clinically not applicable.

In the majority of previously reported studies on cardiac stem cells, the differentiation of the pluripotent stem cells into myocardial cells is judged based on only the presence or absence of cell beating, and there are few studies where the differentiation into myocardial cells is accurately judged by discriminating myocardial cells from undifferentiated skeletal myoblasts. Accordingly, the conventionally reported cardiac stem cells are often skeletal myoblasts or cells that cannot be differentiated into muscle cells, and are scarcely clinically applicable at present.

With such conventional techniques given as a background, it is desired to establish a regeneration therapy capable of essential regeneration of the cardiac muscle by isolating stem cells at high purity capable of essential regeneration into myocardial cells and utilizing the stem cells to transplant myocardial cells in disordered myocardial regions.

Patent Document 1: International Publication No. 03/80798

Patent Document 2: International Publication No. 03/27281

### DISCLOSURE OF THE INVENTION

### Problems to be Solved by the Invention

An object of the present invention is to solve the problems in the conventional techniques described above. Specifically, an object of the present invention is to isolate a pluripotent stem cell at high purity capable of differentiation into at least a myocardial cell to regenerate the cardiac muscle. Another object of the present invention is to provide inventions utilizing the pluripotent stem cell, specifically a method of treating cardiac diseases, a pharmaceutical composition useful for various diseases, and a method of screening for a substance capable of differentiation induction or amplification of the pluripotent stem cell.

### Means for Solving the Problems

The present inventors made extensive study to solve the problems, and as a result they found that a pluripotent stem cell can be obtained at high purity by culturing in a culture medium (serum free culture medium) a skeletal muscle tissue-derived cell obtained by enzymatically treating a collected skeletal muscle tissue and isolating a colony that is floating in the culture medium. Further, the inventors confirmed through an electrophysiological means that the resulting pluripotent stem cell can be differentiated into at least a pulsatile myocardial cell. On the basis of these findings, the present invention was completed by further examination.

That is, the present invention relates to:

1. An isolated pluripotent stem cell derived from a mammalian skeletal muscle tissue, which is c-met-negative, Pax-7-negative, Myf-5-negative, MyoD-negative, Myogenin-negative, and M-cadherin-negative.
2. The pluripotent stem cell according to the above-mentioned 1, which is CD105- and CD90-positive and c-kit- and CD45-negative.
3. The pluripotent stem cell according to the above-mentioned 1 or 2, which is Sox-2-positive, Cripto-positive, Nanog-positive, Oct-4-positive, Bmi-1-positive, and Brcp-positive.
4. The pluripotent stem cell according to any of the above-mentioned 1 to 3, which is a pluripotent stem cell having an ability to be differentiated into one or more cell(s) selected from the group consisting of a skeletal muscle cell, a smooth muscle cell, a myocardial cell, a blood cell, a vascular endothelial cell, a fat cell, a cartilage cell, an osteoblastic cell, and a neural cell.
5. The pluripotent stem cell according to any of the above-mentioned 1 to 3, which is a pluripotent stem cell having an ability to be differentiated at least into a pulsatile myocardial cell.
6. The pluripotent stem cell according to any of the above-mentioned 1 to 5, wherein the mammal is at least one member selected from the group consisting of a human, rat, mouse, sheep, swine, canine and simian.
7. The pluripotent stem cell according to any of the above-mentioned 1 to 6, which is obtained through the steps consisting of:

(i) collecting a skeletal muscle tissue from a mammal and enzymatically treating the obtained skeletal muscle tissue to prepare a skeletal muscle tissue-derived cell;

(ii) culturing the obtained skeletal muscle tissue-derived cell in a culture medium containing an epidermal growth factor and a fibroblast growth factor; and

(iii) selecting and separating a colony that is floating in the culture medium.

8. The pluripotent stem cell according to the above-mentioned 7, wherein the step (iii) is a step of selecting and separating only one colony which among colonies floating in the culture medium, is formed by proliferation of a single cell.
9. A pluripotent stem cell group composed of the pluripotent stem cell of any of the above-mentioned 1 to 8 and obtained by proliferation of a single cell.
10. The pluripotent stem cell group according to the above-mentioned 9, which is obtained through the following steps:

(i) collecting a skeletal muscle tissue from a mammal and enzymatically treating the obtained skeletal muscle tissue to prepare a skeletal muscle tissue-derived cell;

(ii) culturing the obtained skeletal muscle tissue-derived cell in a culture medium containing an epidermal growth factor and a fibroblast growth factor;

(iii) selecting and separating a colony that is floating in the culture medium; and

(iv) proliferating cells forming the colony separated in the step (iii) above.

11. The pluripotent stem cell group according to the above-mentioned 10, wherein the step (iii) comprises selecting a colony formed by proliferation of a single cell from the colonies floating in the culture medium, and then separating the colony in such a manner that only the single colony is present.
12. A method of proliferating the pluripotent stem cell of any of the above-mentioned 1 to 7, which comprises culturing the pluripotent stem cell in a follistatin-containing culture medium.
13. Use of follistatin for proliferating the pluripotent stem cell of any of the above-mentioned 1 to 7.
14. A method of isolating the pluripotent stem cell according to any of the above-mentioned 1 to 8, comprising the steps consisting of:

(i) collecting a skeletal muscle tissue from a mammal and enzymatically treating the obtained skeletal muscle tissue to prepare a skeletal muscle tissue-derived cell;

(ii) culturing the obtained skeletal muscle tissue-derived cell in a culture medium containing an epidermal growth factor and a fibroblast growth factor; and

(iii) selecting and separating a colony that is floating in the culture medium.

15. The method of isolating a pluripotent stem cell group according to the above-mentioned 14, wherein the step (iii) comprises selecting a colony formed by proliferation of a single cell from the colonies floating in the culture medium, and then separating the colony in such a manner that only the single colony is present.
16. A process for preparing the pluripotent stem cell group of the above-mentioned 9, comprising the steps consisting of:

(i) collecting a skeletal muscle tissue from a mammal and enzymatically treating the obtained skeletal muscle tissue to prepare a skeletal muscle tissue-derived cell;

(ii) culturing the obtained skeletal muscle tissue-derived cell in a culture medium containing an epidermal growth factor and a fibroblast growth factor;

(iii) selecting and separating a colony that is floating in the culture medium; and

(iv) proliferating cells forming the colony separated in the step (iii) above.

17. The process for preparing a pluripotent stem cell group according to the above-mentioned 16, wherein the step (iii) comprises selecting a colony formed by proliferation of a single cell from the colonies floating in the culture medium, and then separating the colony in such a manner that only the single colony is present.
18. A therapeutic method for a tissue or organ disease, which comprises transplanting the pluripotent stem cell of any of the above-mentioned 1 to 8 or a cell differentiated from the stem cell into a patient's tissue or organ.
19. The therapeutic method according to the above-mentioned 18, wherein the disease to be treated is a cardiac disease.
20. A pharmaceutical composition comprising the pluripotent stem cell of any of the above-mentioned 1 to 8.
21. The pharmaceutical composition according to the above-mentioned 20, which is an agent for regenerating a tissue or a cell.
22. The pharmaceutical composition according to the above-mentioned 20, which is an agent for treating an organ dysfunction.
23. Use of the pluripotent stem cell of any of the above-mentioned 1 to 8 in production of a pharmaceutical composition for treatment of a tissue or organ disease.
24. Use according to the above-mentioned 23, wherein the pharmaceutical composition is an agent for regenerating an organ or a cell.
25. Use according to the above-mentioned 23, wherein the pharmaceutical composition is an agent for treatment of a cardiac disease.
26. A method of screening for a substance that induces differentiation of the pluripotent stem cell of any of the above-mentioned 1 to 8 into various types of cells, which comprises the following steps:
(a) contacting a test substance with the pluripotent stem cell and culturing the cell; and
(b) observing whether differentiation induction of the pluripotent stem cell occurs or not, and depending on the result, separating the test substance.
27. A method of screening for a substance amplifying the pluripotent stem cell of any of the above-mentioned 1 to 8, which comprises the following steps:
(a) contacting a test substance with the pluripotent stem cell and culturing the cell; and
(b) observing whether amplification of the pluripotent stem cell occurs or not, and depending on the result, separating the test substance.

### EFFECTS OF THE INVENTION

Any conventionally reported skeletal muscle tissue-derived pluripotent stem cells have low purity and inevitably contain different types of cells such as skeletal myoblasts and fibroblasts, and are thus not clinically applicable in cell transplantation. For example, when stem cells contaminated with skeletal myoblasts are transplanted in the heart, there arises the clinical problem of generation of serious arrhythmogenic activity.

On the other hand, the pluripotent stem cell of the present invention is isolated by cloning a single cell present in a skeletal muscle tissue, thus minimizing contamination with different types of cells and attaining high purity not achieved by the conventionally reported stem cells. Accordingly, when the pluripotent stem cell of the present invention is used, cell transplantation in patients with cardiac diseases or the like can be carried out safely without side effects caused by transplantation of different cells.

The skeletal muscle tissue-derived pluripotent stem cell of the present invention can be subcultured while maintaining its undifferentiated state for a long time and is thus highly useful with clinical practicability.

The pluripotent stem cell of the present invention is excellent particularly in an ability to be differentiated into a myocardial cell and can thus provide a new therapeutic method by cell transplantation in a patient with serious heart failure who cannot but rely on cardiac transplantation, and its usefulness is extremely high. It has been elucidated that differentiation of the pluripotent stem cell into a myocardial cell in cell plantation therapy of a patient with heat failure is based on the mechanism of both differentiation via cell fusion with a host myocardial cell and positive differentiation into a myocardial cell without cell fusion with a host myocardial cell.

### BRIEF DESCRIPTION OF THE DRAWINGS

FIG. 1 shows cells or cell colonies observed when mouse skeletal muscle tissue-derived cells were cultured in (2) in Example 1. In FIGS. 1, A and B show stem cell colonies floating in a culture medium. In FIG. 1, C shows fibroblasts with a small nucleus extending in an elongated shape, adhering to the bottom of a culture dish. In FIG. 1, D shows skeletal myoblasts spherically forming a population adhering to the bottom of a culture dish.

FIG. 2 is photographs of floating cells or colonies observed before and after wild-type mouse skeletal muscle tissue-derived cells and GFP-expressing mouse skeletal muscle tissue-derived cells were mixed and cultured in (4) in Example 1. The upper left photograph is a phase-contrast micrograph of floating cells at the start of culture on a non-coating 10-cm cell culture dish; the lower left photograph is a fluorescence micrograph of the same visual field as in the upper left photograph; the upper right photograph is a phase-contrast micrograph of floating colonies after culture on a 10-cm fibronectin coating dish; and the lower right photograph is a fluorescence micrograph of the same visual field as in the upper left photograph.

FIG. 3 is photographs showing the result of observation of expression of bromodeoxyuridine (BrdU) in the stem cells isolated in (3) in Example 1. The lower left photograph shows the result (fluorescence micrograph) of observation of expression of BrdU in the above stem cells; the upper left photograph is a phase-contrast micrograph of the same visual field as in the lower left photograph; the lower right photograph shows the result (fluorescence micrograph) of observation of expression of BrdU in marginal cells proliferated by culture for 5 days from the stem cells; and the upper right photograph is a phase-contrast micrograph of the same visual field as in the lower right photograph.

FIG. 4 shows the results of observation with time of proliferation characteristics of the stem cells isolated in (3) in Example 1 and the adherent cells collected in (3) in Example 1. In FIG. 4, A is a micrograph showing the state of the stem cells on day 3 of culture; B, the state of the stem cells on day 7 of culture; and C, the state of the stem cells after the 3^{rd} passage of the stem cells. In FIG. 4, D shows a micrograph showing the state of growth of the adherent cells; E, the state of formation of myotube tubes from the adherent cells; and F, the state of differentiation of the adherent cells into skeletal muscle cells.

FIG. 5 shows the results of analysis of expression of ES cell markers (Bcrp, Bmi-1, Cripto, UTF-1, Nanog, Oct-4, HNF-3β, Brachyury, Sox2 and β-actin) in the stem cells (colonies) isolated in (3) in Example 1. In FIG. 5, (number of positive colonies/number of colonies analyzed) is shown in parentheses following each marker.

FIG. 6 shows the results of FACS analysis of various cell surface antigens (CD34, Sca-1, CD45, CD90, CD105, CD117, CD13, CD31 and CD38) of the mouse-derived stem cells isolated in (3) in Example 1. In FIG. 6, the analysis result of the mouse-derived stem cells is shown in the red line, while the analysis result of the control (cells with no label) is shown in the blue line.

FIG. 7 shows the results of PCR analysis of expression of myoblast C2C12 and skeletal myoblast markers (Pax-7, Myf-5, c-met, M-cadherin, MyoD and Myogenin) in the stem cells isolated in (3) in Example 1.

FIG. 8 shows images observed by staining CD34 red with Alex Fluor 555, staining Myf-5 green with Alex Fluor 488, and staining intracellular nuclei blue with DAPI (4′,6-diamino-2-phenylindole), in the stem cells isolated in (3) in Example 1 and the adherent cells collected in (3) in Example 1. In FIG. 8, A is an image of the adherent cells stained with CD34; B is an image of the adherent cells stained with Myf-5 in the same visual field as in A; C is an image of the adherent cells stained with DAPI in the same visual field as in A; D is a composite image of A to C; E is an image of the stem cells stained with CD34; F is an image of the stem cells stained with Myf-5 in the same visual field as in E; G is an image of the stem cells stained with DAPI in the same visual field as in E; and H is a composite image of E to G.

FIG. 9 shows the results of observation of local sites of the stem cells and skeletal myoblasts in Example 2. In FIG. 9, A shows skeletal myoblasts present under the cell basement membrane, and B shows stem cells localized in the interstitial tissue. In A and B, laminine staining gives a green color, DAPI staining gives a blue color, and CD34 staining gives a red color. In FIG. 9, C shows skeletal myoblasts (yellow arrow) present under the cell basement membrane and stem cells (white arrow) present in the interstitial tissue. In C, laminine staining gives a red color, DAPI staining gives a blue color, and Myf-5 staining gives a green color.

FIG. 10 is micrographs showing differentiation of the stem cells isolated in (3) in Example 1 into myocardial cells. In A in FIG. 10, the upper left photograph shows the differentiation of the stem cells into prometaphase myocardial cells; the upper right photograph shows the differentiation of the stem cells into early anaphase myocardial cells; the lower left photograph shows the differentiation of the stem cells into late anaphase myocardial cells; and the lower right photograph shows the differentiation of the stem cells into late telophase myocardial cells. In A in FIG. 10, DAPI staining gives a blue color, and cardiac-specific troponin-I staining gives a green color. In FIG. 10, B is micrographs showing that recombinant stem cells transformed with a GFP gene controlled by an alpha-myocardial heavy chain promoter are differentiated into myocardial cells, and in B, the upper left photograph is an image observed under a phase-contrast microscope; the upper right photograph is an image observed in the same visual field as in the upper left photograph under a fluorescence microscope; and the lower right photograph is an enlarged image of the circled part in the upper right photograph.

FIG. 11 is profiles showing the PCR analysis results of expression of various markers recognized in myocardial cells (various myocardial transcription factors, structural proteins, myocardium-binding proteins, and calcium ion channels) in the stem cells isolated in (3) in Example 1 before induction of differentiation into myocardial cells, and in the cells on day 14 after starting of differentiation induction.

FIG. 12 is micrographs showing a differentiated or undifferentiated cellular state exhibited by the stem cells isolated in (3) in Example 1 and skeletal myoblasts. In FIG. 12, A shows a pulsatile myotube state formed in a sheet shape by proliferation and differentiation of the skeletal myoblasts; B shows an undifferentiated state of the skeletal myoblasts in a multinucleate cell state showing rhythmical beating; C shows a differentiated state of the stem cells, that is, a state of pulsatile myocardial cells in a mononuclear or binuclear state; and D show a state of regularly beating mononuclear myocardial cells into which the stem cells were differentiated.

FIG. 13 shows the results of electrophysiological examination of the pulsatile characteristics of the stem cells isolated in (3) in Example 1 and skeletal myoblasts. In FIG. 13, A shows an amplitude pattern of action potential of the skeletal muscle cells; B shows an amplitude pattern of action potential of the skeletal muscle cells before and after addition of isoproterenol; C shows an amplitude pattern of action potential of myocardial cells formed through differentiation induction from the stem cells; and D shows an amplitude pattern of action potential, before and after addition of isoproterenol, of myocardial cells formed through differentiation induction from the stem cells.

FIG. 14 shows the results of observation of various types of cells into which the stem cells obtained in (3) in Example 1 were differentiated. In FIG. 14, A shows vascular smooth muscle cells into which the stem cells were differentiated; B shows endothelial cells into which the stem cells were differentiated; C shows glia cells into which the stem cells were differentiated; D shows neural cells into which the stem cells were differentiated; E shows fat cells into which the stem cells were differentiated; F shows epithelial cells into which the stem cells were differentiated; G shows bone cells into which the stem cells were differentiated; and H shows skeletal muscle cells into which the stem cells were differentiated.

FIG. 15 is photographs showing an engrafted and differentiated state of stem cells in a mouse heart muscle in Example 5 wherein skeletal muscle tissue-derived stem cells from a mouse expressing LacZ were transplanted in a mouse infarcted heart muscle. In FIGS. 15, A, C and E show the results of staining of LacZ; B shows the result of staining with DAPI and staining using cardiac-specific troponin-I, in the same visual field as in A; D shows the result of staining with DAPI and staining using CD31, in the same visual field as in C; and F shows the result of staining with DAPI and staining using α-smooth muscle-MHC, in the same visual field as in E.

FIG. 16 shows the results of observation of the state of human skeletal muscle tissue-derived stem cells in Example 6. In FIG. 16, A shows stem cell groups (colonies) which after culture of human skeletal muscle tissue-derived cells, float in a culture medium and show a proliferation ability, and B is an enlarged image of A. C shows an isolated human skeletal muscle tissue-derived stem cell colony. D shows concentric growth of the isolated human skeletal muscle tissue-derived stem cell colony after 5 days of culture.

FIG. 17 shows the results of analysis of expression of ES cell markers (Nanog, Oct-4, Rex1, Brachyury and Sox 2) in the obtained human skeletal muscle tissue-derived stem cells in Example 6.

FIG. 18 shows the results of FACS analysis of various cell surface antigens (CD56, CD34, CD45, CD117, CD90, CD105, CD31 and CD38) of the human skeletal muscle tissue-derived stem cells in Example 6. In FIG. 18, the analysis result of the human skeletal muscle tissue-derived stem cells is shown in the red line, while the analysis result of the control (cells with no label) is shown in the blue line.

FIG. 19 shows the results of observation of various types of cells into which the human skeletal muscle tissue-derived stem cells obtained in Example 6 were differentiated in Example 7. In FIG. 19, A shows myocardial cells into which the stem cells were differentiated; B shows skeletal muscle cells into which the stem cells were differentiated; C shows smooth muscle cells into which the stem cells were differentiated; and D shows endothelial cells into which the stem cells were differentiated.

FIG. 20 is photographs showing an engrafted state of stem cells in a mouse heart muscle in Example 8 wherein the human skeletal muscle tissue-derived stem cells obtained in Example 6 were transplanted in a mouse infarcted heart muscle. In FIG. 20, A shows the results of staining myocardial troponin-I-positive myocardial cells red and staining human-derived nuclei green; B shows the results of staining myocardial troponin-I-positive myocardial cells red and staining all nuclei white with DAPI, in the same visual field as in A; C shows the results of staining alpha-smooth muscle myosin heavy chain-positive smooth muscle cells red, staining human-derived nuclei green, and staining all nuclei blue with DAPI; and D shows the results of staining CD31-positive endothelial cells red, staining human-derived nuclei green, and staining all nuclei blue with DAPI.

FIG. 21 shows the results of Example 9. A shows the number (black circle) of colonies of mouse skeletal muscle-derived stem cells obtained from 6-, 12- and 24-week-old mice and the proportion (white circle) of Myt5-positive satellite cells. B shows cell population doubling measured when the skeletal muscle-derived stem cells were cultured with a passage number of 120 for 400 days.

FIG. 22 shows the results of confirmation of the characteristics of mouse skeletal muscle-derived stem cells cultured with a passage number of 120 for 400 days. In A, the left photograph shows the state of initially formed stem cell colonies (passage number, 0); the middle photograph shows the state of the cells which were subjected to adhesion culture with a passage number of 120; and the right photograph shows the state of the stem cells which while forming colonies, were proliferated after culture with a passage number of 120 by exchanging the culture medium with a serum-free culture medium. B shows the results of staining, with Sca-1, CD34 and Nestin, of mouse skeletal muscle-derived stem cell colonies cultured with a passage number of 120 for 400 days. C shows the results of PCR analysis of expression of ES cell markers Nanog and Oct-4.

FIG. 23 is profiles showing the results of examination of the influence of follistatin on culture of skeletal muscle-derived stem cells. A shows the results of Western blotting analysis of expression of follistatin and myostatin in ES cells (mES), the initial (passage number 0) skeletal muscle-derived stem cells (primary sphere) obtained in Example 9, the skeletal muscle-derived stem cells (tertiary sphere) obtained in Example 9 and passaged 3 times, mouse myoblasts (C2C12), satellite cells, adult human muscle-derived adult cells (adult muscle), and adult heart muscle-derived adult cells (adult heart). B shows the results of PCR analysis of expression of p21, Cdk2 and Rb in skeletal muscle-derived stem cells cultured in a culture medium supplemented with 0.5 μg/mL or 1.0 μg/mL of myostatin. In B, (−) shows the result of skeletal muscle-derived stem cells cultured in a follistatin-free culture medium.

FIG. 24 is photographs showing the state of a floating cell (sphere) and adherent cells (satellite cells) observed when skeletal muscle-derived stem cells from a myostatin-deficient mouse (mstn−/−) and skeletal muscle-derived stem cells from a GFP-expressing mouse (GFP-Tg) were co-cultured in Example 11. In FIG. 24, the results of observation under a phase-contrast microscope and a fluorescence microscope are shown in the columns “phase” and “GFP”, respectively.

FIG. 25A is photographs showing the state of a floating cells (sphere) and adherent cells (satellite cells) observed when skeletal muscle-derived stem cell colonies were cultured in the presence or absence of follistatin in Example 11. Among the observation results of the state of the floating cell (sphere), the upper photograph shows the result of observation under a phase-contrast microscope, while the lower photograph shows the result of observation under a fluorescence microscope. FIG. 25B shows the results of Western blotting analysis of expression with time of p-smad2/3, smad2/3, p-smad1/5/8, smad1/5/8, p21, Cdk2 and Rb in skeletal muscle-derived stem cells, which after cultured in the presence or absence of follistatin, constituted colonies.

FIG. 26A shows the results of PCR analysis of expression of Nodal, Activin (Act A and Act B) and GDF11 in skeletal muscle-derived stem cells in Example 11. FIG. 26B shows the results of PCR analysis of expression of activin receptors and ALK2, 3, 4, 5 and 7 in skeletal muscle-derived stem cells.

FIG. 27 is photographs showing the test results in Example 12, that is, the state of engraftment of stem cells in a mouse heart muscle in Example 12 wherein skeletal muscle-derived stem cells obtained from a LacZ reporter mouse were transplanted in a mouse infarcted heart muscle. The upper photographs show that the transplanted, skeletal muscle-derived stem cells are fused with cells of a host heart muscle and differentiated into myocardial cells. The middle photographs show that the transplanted, skeletal muscle-derived stem cells are also differentiated positively into myocardial cells without cell-cell fusion. The lower photographs show that a part of the transplanted, skeletal muscle-derived stem cells is differentiated into non-myocardial cells.

FIG. 28 shows the results of analysis of cardiac functions in an infarcted heart muscle transplanted with skeletal muscle-derived stem cells in Example 13. A shows typical echocardiograms of a group of C57Bl/6J mice in which infarcted heart muscles were not generated (sham group), a group of mice with myocardial infraction injected with PBS (I+PBS group), and a group of mice with myocardial infraction transplanted with skeletal muscle-derived stem cells (MI+sk+MSC group). B shows the results of measurement of left ventricular end-diastolic diameter (LVDd), fractional shortening (FS) and left ventricular diastolic performance (E/A) in the sham group (gray, left), the I+PBS group (middle, while) and the MI+sk+MSC group (black, right).

### BEST MODE FOR CARRYING OUT THE INVENTION

Hereinafter, the present invention is described in detail.

The pluripotent stem cell of the present invention is an isolated pluripotent stem cell derived from a mammalian skeletal muscle tissue, which is c-met-negative, Pax-7-negative, Myf-5-negative, MyoD-negative, Myogenin-negative, and M-cadherin-negative, and is distinguished clearly from skeletal myoblasts.

Specific examples of the pluripotent stem cell of the present invention include those cells which are CD105- and CD90-positive and c-kit- and CD45-negative with respect to cell surface antigen characteristics. The pluripotent stem cell of the present invention is exemplified by CD13- and CD38-negative or weakly-positive cells. The pluripotent stem cell of the present invention is characterized by being CD34-negative in the case of the human-derived stem cells and being CD34-positive in the case of the mouse-derived stem cells.

Specific examples of the preferred pluripotent stem cell of the present invention include Sox-2-positive, Cripto-positive, Nanog-positive, Oct-4-positive, Bmi-1-positive, and Brcp-positive cells.

The pluripotent stem cell of the present invention has, together with a proliferation ability, an ability to be differentiated into one or more cells selected from the group consisting of a skeletal muscle cell, a smooth muscle cell, a myocardial cell, a blood cell, a vascular endothelial cell, a fat cell, a cartilage cell, an osteoblastic cell, and a neural cell. The pluripotent stem cell of the present invention preferably has an ability to be differentiated into all the cells mentioned above.

In one aspect, the pluripotent stem cell of the present invention is characterized by having an ability to be differentiated at least into a pulsatile myocardial cell.

The mammal from which the pluripotent stem cell of the present invention is derived is not particularly limited, and includes for example a human, rat, mouse, sheep, swine, canine and simian. The pluripotent stem cell of the present invention when used for treatment of human cardiac diseases is preferably human-derived.

When the pluripotent stem cell of the present invention is derived from a mammal skeletal muscle tissue, the skeletal muscle tissue may be derived from any body sites and can be exemplified by skeletal muscle tissues for example in the leg, arm, shoulder, neck, back, hip, face/head, breast and belly.

Hereinafter, the pluripotent stem cell of the present invention is described in “A. Method of Isolating the Pluripotent Stem Cell of the Invention”, “B. Culture (Proliferation) of the Pluripotent Stem Cell of the Invention”, “C. Induction of Differentiation of the Pluripotent Stem Cell of the Invention into the Objective Cell”, “D. Therapeutic Method for Diseases”, E. Pharmaceutical Composition” and “F. Screening Method”. According to “A. Method of Isolating the Pluripotent Stem Cell of the Invention” and “B. Culture (Proliferation) of the Pluripotent Stem Cell of the Invention”, the pluripotent stem cell of the present invention is isolated and cultured, whereby a cell group (cell population) containing the pluripotent stem cells of the present invention at high purity can be obtained.

### A. Method of Isolating the Pluripotent Stem Cell of the Invention

1. Acquisition of a Cell Derived from a Skeletal Muscle Tissue

First, a skeletal muscle tissue is collected from a mammal, and the obtained skeletal muscle tissue is enzymatically treated, whereby a skeletal muscle tissue-derived cell is acquired (step (i)).

The collection of a skeletal muscle tissue from a mammal is carried out by excising a skeletal muscle tissue by a usual surgical method. Preferably, the excised skeletal muscle cell is deprived of tissues as much as possible (for example, blood vessels, nerves, tendon, ligament, bone tissue and the like) other than the skeletal muscle tissue, prior to enzyme treatment. To increase the efficiency of enzyme treatment, the collected skeletal muscle tissue is preferably cut thin into fragments of about 1 mm³ or less prior to enzyme treatment.

The skeletal muscle tissue is subjected to enzyme treatment in a suitable buffer, thereby giving the objective skeletal muscle tissue-derived cell. The buffer used herein is not particularly limited insofar as the cell and enzyme are not adversely affected, and examples of such buffer include Hanks' Balanced Salt Solution (manufactured by GIBCO) containing 1 wt % of penicillin-streptomycin and 2 mM of L-glutamine.

The enzyme treatment is carried out by using a generally used enzyme in separating the cell from the biomedical tissue fragment. Specific examples include proteases such as collagenase, trypsin, chymotrypsin and pepsin, among which collagenase is preferable. Specific examples of such collagenase include collagenase type 2 (205 U/mg; manufactured by Worthington Biochemical Corporation). In this specification, 1 U of collagenase refers to the amount of the enzyme which can release 1 μM L-leucine from collagen at pH 7.5 at 35° C. in 5 hours.

The enzyme treatment conditions are not particularly limited either, and by way of example, the following enzyme treatment conditions can be mentioned.

Enzyme concentration: For example, when collagenase type 2 (205 U/mg; manufactured by Worthington Biochemical Corporation) is used, the concentration is usually 0.2 to 0.6% by weight, preferably about 0.4% by weight; or the concentration is usually 3075 to 9225 U, preferably about 6150 U, per 2 g of skeletal muscle tissue.
Treatment temperature: Usually a temperature of about 37° C.
Treatment time: Usually 30 to 60 minutes, preferably a time of about 45 minutes.

By performing the enzyme treatment in this way, a skeletal muscle tissue-derived cell is released from the skeletal muscle tissue, and after the enzyme treatment, the skeletal muscle tissue-derived cell is separated by a known means such as centrifugation, whereby the skeletal muscle tissue-derived cell can be obtained. To the skeletal muscle tissue-derived cell thus obtained is desirably added a culture medium suitable for the growth of the cell. Such culture medium is exemplified by, for example, Dulbecco's modified Eagle culture medium (DMEM) containing 10% by volume of fetal bovine serum (FBS) and 1% by volume of penicillin-streptomycin (mixture of 5000 U/ml of penicillin and 5000 μg/ml of streptomycin sulfate).

The skeletal muscle tissue-derived cell thus obtained may be subjected if necessary to filtration treatment or the like to remove components other than the cell.

### 2. Culture of the Skeletal Muscle Tissue-Derived Cell

Then, the skeletal muscle tissue-derived cell obtained as described above is cultured in a culture medium containing an epidermal growth factor (EGF) and a fibroblast growth factor (FGF) (step (ii)).

When the skeletal muscle tissue-derived cells obtained in the step (i) above are bound or adhered to one another, the skeletal muscle tissue-derived cells may be subjected further to enzyme treatment prior to culture, thereby cancelling the binding or adhesion of the cells. A specific method of such enzyme treatment is not particularly limited and can be carried out by a known method using protease or the like. By way of example, the enzyme treatment is exemplified by a method that involves treating the skeletal muscle tissue-derived cell group with a solution containing 0.05 wt % of trypsin and 0.53 mM of EDTA at 37° C. for about 10 minutes. After this enzyme treatment, a protease inhibitor is desirably added thereto to inactivate the protease activity before the cells are subjected to the step (iii).

The culture medium used in this step may be a culture medium comprising an epidermal growth factor and a fibroblast growth factor added to a medium used in usual cell culture (suspension culture). Preferable examples of the culture medium include a culture medium containing about 2 vol % of B27 supplement (manufactured by GIBCO) (for example, DMEM/F12 culture medium or the like) to which an epidermal growth factor and a fibroblast growth factor are added. If necessary, the medium used in this step may contain antibiotics such as streptomycin, kanamycin and penicillin and amino acids such as glutamic acid.

The proportions of an epidermal growth factor and a fibroblast growth factor compounded in the culture medium used in this step are exemplified by, for example, about 20 ng/ml of the epidermal growth factor and about 40 ng/ml of the fibroblast growth factor.

In this step, the density of cells at the start of culture is set desirably at 1×10⁴ to 4×10⁴ cells/ml, preferably about 2×10⁴ cells/ml, in order to perform culture.

Though not intended to limit this step, it is desired that the skeletal muscle tissue-derived cells obtained in the step (i) above are cultured first in a non-coating culture dish, and then the resulting culture medium is transferred to, and cultured in, a culture dish coated with fibronectin or the like.

Culture in this step is carried out usually at 37° C. under 5% CO₂, usually for 7 to 21 days, preferably 10 to 14 days.

### 3. Isolation of the Skeletal Muscle Tissue-Derived Pluripotent Stem Cell

In the culture medium obtained in the step (ii), colonies formed by proliferation of the skeletal muscle tissue-derived pluripotent stem cells occur in a floating state, while fibroblasts and skeletal myoblasts derived from the skeletal muscle tissue occur in a state adhering to the bottom of a culture vessel. Accordingly, the colonies formed through cell proliferation are selected and separated from the culture medium obtained in the step (ii) above, whereby the skeletal muscle tissue-derived pluripotent stem cells can be isolated at high purity (step (iii)). Herein, the “colony formed by proliferation of a single cell” in a floating state in the culture medium obtained in the step (ii) above is selected so as to be unmixed with other cells and is separated such that the single colony is present, whereby a cell mass derived from the single pluripotent stem cell can be isolated.

A method of selecting and separating the colony formed through cell proliferation in this step is not particularly limited, and for example, a method of collecting the colony with a micropipette under a microscope can be mentioned.

In this step, it is desirable that the objective colony is selected and separated accurately by discriminating the colony formed through cell proliferation from a cell mass in which skeletal muscle tissue-derived cells merely adhere to one another. A specific method of selecting and separating the objective colony accurately in this step includes the following method: first, in the step (ii) above, the skeletal muscle tissue-derived cell is cultured in the coexistence of a pigment-expressing cell (for example, a green fluorescence protein (GPF)-expressing cell). In the step (iii), a cell mass not containing the pigment-expressing cell is selected and separated from the culture medium as the objective colony. In this method, it can be judged that among floating cell masses, cell masses containing the pigment-expressing cell are formed by mere adhesion of the cells, while cell masses not containing the pigment-expressing cell are colonies formed by proliferation of the pluripotent stem cell.

The fact that the colony obtained in this step is composed of the pluripotent stem cell of the present invention can be confirmed by measuring expression of a marker of the stem cell, if necessary after proliferation by culture described later.

### B. Culture (Proliferation) of the Pluripotent Stem Cell of the Invention

The colony of the single pluripotent stem cell isolated as described above is cultured in a culture medium containing an epidermal growth factor and a fibroblast growth factor, and the pluripotent stem cell is proliferated, whereby the high-purity pluripotent stem cell group (cell population) derived from the single pluripotent stem cell can be obtained. The culture medium used in proliferation of the pluripotent stem cell of the present invention includes a culture medium comprising an epidermal growth factor, a fibroblast growth factor, and a leukemia inhibitory factor (LIF) added to a culture medium used in usual cell culture (suspension culture). If necessary, the culture medium used in this step may contain antibiotics such as streptomycin, kanamycin and penicillin and amino acids such as glutamic acid. The culture medium can be exemplified more specifically by a medium comprising an epidermal growth factor, a fibroblast growth factor and a leukemia inhibitory factor added to a culture medium containing about 2 vol % of fetal bovine serum and about 1 vol % of L-glutamine (20 mM)-penicillin (10000 units/mL)-streptomycin (10 mg/mL) [for example, Advanced DMEM/F12 (manufactured by GIBCO) or the like].

The proportions of an epidermal growth factor, a fibroblast growth factor and a leukemia inhibitory factor in the culture medium used in this step are exemplified by, for example, about 20 ng/ml of the epidermal growth factor, about 10 ng/ml of the fibroblast growth factor, and about 10 ng/ml of the leukemia inhibitory factor.

When follistatin is further added to the culture medium described above, the growth rate of the pluripotent stem cell of the present invention can be increased selectively and appropriately. Although the amount of follistatin added to the culture medium is not particularly limited, the concentration is preferably about 652 ng/mL, for example.

In culture of the pluripotent stem cell in the follistatin-containing culture medium, the pluripotent stem cells can be proliferated selectively by culturing them at a density of about 20 cells/μl at the start of culture, for example at 37° C. under 5% CO₂, usually for about 10 to 14 days.

The pluripotent stem cells are advantageous in that they can be subcultured while maintaining their undifferentiated state for a long time, and regardless of whether follistatin is added or not, the culture period can also be set 400 days or longer if necessary.

C. Induction of Differentiation of the Pluripotent Stem Cell of the Invention into the Objective Cell

The method of inducing differentiation of the pluripotent stem cells into various types of cells including myocardial cells includes, for example, a method wherein the proliferated pluripotent stem cells are cultured in a culture medium containing an inducer for inducing differentiation of the stem cells into objective cells.

Particularly, for induction of differentiation of the pluripotent stem cells into myocardial cells, dexamethasone can be preferably used as the inducer. Hereinafter, the method of differentiation induction is described in detail by reference to induction of differentiation into myocardial cells.

In the culture medium used in induction of differentiation into myocardial cells, the proportion of dexamethasone added is not particularly limited insofar as the induction of differentiation into myocardial cells is feasible, but usually, dexamethasone may be contained in a proportion of about 1×10⁻⁸ mol/l in the culture medium.

Although the culture medium used in induction of differentiation into myocardial cells is not particularly limited, an MEM culture medium (minimum essential medium, manufactured by GIBCO) supplemented with dexamethasone is mentioned as a preferable culture medium. Similarly to the culture medium used in proliferation of the pluripotent stem cell, the culture medium may contain antibiotics such as streptomycin, kanamycin and penicillin and other components such as insulin, transferrin and selenium-X, and the like, if necessary.

Using the culture medium described above, the pluripotent stem cell is cultured usually at 37° C. under 5% CO₂, usually for 7 to 21 days, preferably 14 days, thereby inducing differentiation of the pluripotent stem cells in a predetermined proportion into myocardial cells.

For induction of differentiation of the pluripotent stem cells into various types of cells other than myocardial cells, the inducer used and its concentration in the culture medium are exemplified as follows: for example, about 10 ng/ml of a platelet-derived growth factor (PDGF-BB) in the case of induction of differentiation into a vascular smooth muscle cell; about 10 ng/ml of a vascular endothelial growth factor (VEGF) in the case of induction of differentiation into an endothelial cell; about 5 mM of β-mercaptoethanol in the case of induction of differentiation into a glia cell; about 10 ng/ml of a brain-derived neural factor (BDNF) in the case of induction of differentiation into a neural cell; 1×ITS-A (insulin transferrin-selenite), about 1×10⁻⁸ M of dexamethasone, and about 0.5 mM of IBMX (3-isobutyl-1-methylxanthine) in the case of induction of differentiation into a fat cell; and about 10 ng/ml of a TGF (transforming growth factor) and about 50 nM of ascorbic acid-2-phosphate in the case of induction of differentiation into a bone cell. In induction of differentiation into these cells, other culture medium components and culture conditions are the same as described above in induction of differentiation into myocardial cells.

### D. Therapeutic Method for Diseases

The pluripotent stem cell of the present invention can be used in regeneration and repair of various tissues or organs. Specifically, in a patient with a disease in a tissue or organ, a therapeutically effective amount of the pluripotent stem cell can be transplanted in a diseased site of the tissue or organ to treat the disease. The “therapeutically effective amount” as used herein can be suitably determined depending on the intended disease and the severity thereof, the age and sex of the patient, or the like, and can be exemplified by, for example, about 1.0×10⁷ to 1.0×10⁹ cells.

Alternatively, the disease can be treated by differentiation of the pluripotent stem cell into the objective cell and then transplanting the differentiated cell in a diseased site of the tissue or organ.

In a therapy using the pluripotent stem cell of the present invention, the intended disease is preferably a cardiac disease. Because the pluripotent stem cell of the present invention is excellent in an ability to be differentiated into a pulsatile myocardial cell, it is thus used particularly preferably in treatment of a cardiac disease among the diseases described above.

The intended cardiac disease includes cardiac diseases with dysfunctions in cardiac muscles or coronary arteries leading to a reduction in contractility and is exemplified specifically by myocardial infarction, dilatative cardiomyopathy, ischemic heart disease, congestive heart failure and the like.

The method of transplanting the pluripotent stem cell includes, for example, a method of injecting the pluripotent stem cell via a catheter into a diseased site of a tissue or organ to be treated and a method of directly injecting the pluripotent stem cell into an incised diseased site of a tissue or organ to be treated.

The method of transplanting cells differentiated from the pluripotent stem cells can be exemplified, for example, by a method which comprises supporting the differentiated cells on a biological sample-absorbing material in a form such as sheet form adapted to the object and attaching the absorbing material onto a diseased site of a tissue or organ to be treated.

In the therapeutic method of the present invention, the pluripotent stem cell collected from a person other than an intended patient with a disease, or a cell differentiated from the pluripotent stem cell, may be used in the patient, but from the viewpoint of inhibiting a rejection reaction, the pluripotent stem cell derived from a heart tissue of the intended patient, or a cell differentiated from the pluripotent stem cell, is preferably used.

The therapeutic method of the present invention includes methods in the following embodiments (I) and (II) as the therapeutic method of cardiac diseases:

(I) A method for treating a disease, which comprises the steps consisting of:

(i) collecting a skeletal muscle tissue from a human and enzymatically treating the obtained skeletal muscle tissue to prepare a skeletal muscle tissue-derived cell;

(ii) culturing the obtained skeletal muscle tissue-derived cell in a culture medium containing an epidermal growth factor and a fibroblast growth factor;

(iii) selecting and separating a colony that is floating in the culture medium obtained in (ii) above;

(iv) proliferating cells forming the colony separated in the step (iii) above; and

(v) transplanting the cells proliferated in the step (iv) in the heart of a patient with a cardiac disease.

(II) A method for treating a disease, which comprises the steps consisting of:

(i) collecting a skeletal muscle tissue from a human and enzymatically treating the obtained skeletal muscle tissue to prepare a skeletal muscle tissue-derived cell;

(ii) culturing the obtained skeletal muscle tissue-derived cell in a culture medium containing an epidermal growth factor and a fibroblast growth factor;

(iii) selecting and separating a colony that is floating in the culture medium obtained in the step (ii) above;

(iv) proliferating cells forming the colony separated in the step (iii) above;

(v) culturing in a dexamethasone-containing culture medium the cells proliferated in the step (iv) above to induce differentiation of the cells into myocardial cells; and

(vi) transplanting the differentiated myocardial cells in the heart of a cardiac patient.

### E. Pharmaceutical Composition

As described above, the skeletal muscle tissue-derived pluripotent stem cell can be used in regeneration and repair of various types of tissues or organs and is useful for treatment of various diseases. Accordingly, the present invention provides a pharmaceutical composition containing the skeletal muscle tissue-derived pluripotent stem cell described above. The pharmaceutical composition may contain a medically or pharmaceutically acceptable carrier and other pharmacological components in addition to the skeletal muscle tissue-derived pluripotent stem cell.

The pharmaceutical composition can be used as an agent for regenerating a tissue or a cell, and is particularly useful as a therapeutic agent for organ dysfunctions. Particularly, the cardiac disease is a preferable therapeutic objective, and specific examples thereof are as described in the above-mentioned “D. Therapeutic Method for Diseases”.

The compounding amount and dosage of the skeletal muscle tissue-derived pluripotent stem cell in the pharmaceutical composition can be determined suitably based on the therapeutically effective amount for the intended disease.

### F. Screening Method

The skeletal muscle tissue-derived pluripotent stem cell can be used to screen for a substance inducing differentiation of the pluripotent stem cell into various types of cells or for a substance amplifying the skeletal muscle tissue-derived pluripotent stem cell. That is, the present invention provides a method of screening for a substance inducing differentiation of the pluripotent stem cell into various types of cells in the following embodiment (III) and a method of screening for a substance amplifying the pluripotent stem cell in the following embodiment (IV).

(III) A method of screening for a substance inducing differentiation of the skeletal muscle tissue-derived pluripotent stem cell into various types of cells, which comprises the steps consisting of:
(a) contacting a test substance with the pluripotent stem cell and culturing the cell; and
(b) observing whether differentiation induction of the pluripotent stem cell occurs or not, and depending on the result, separating the test substance.
(IV) A method of screening for a substance amplifying the skeletal muscle tissue-derived pluripotent stem cell, which comprises the steps consisting of:
(a) contacting a test substance with the pluripotent stem cell and culturing the cell; and
(b) observing whether amplification of the pluripotent stem cell occurs or not, and depending on the result, separating the test substance.

In this screening method, the test substance is not particularly limited, and may be a living body-derived substance, a naturally occurring substance or a synthetic substance.

In this screening method, the concentration of the pluripotent stem cell used, the concentration of a test substance added (concentration of a test substance when contacted with the pluripotent stem cell), culture conditions, and the like can be established suitably depending on the type of a test substance and the object of screening.

### EXAMPLES

Hereinafter, the present invention is described in detail by reference to the Examples and the like, but the present invention is not limited thereto.

### Example 1 Acquisition of Mouse Skeletal Muscle Tissue-Derived Stem Cell

### (1) Acquisition of Mouse Skeletal Muscle Tissue-Derived Cell

Six- to eight-week-old female C57Bl/6J mice (available from Shimizu Laboratory Supplies Co., Ltd.) (hereinafter referred to sometimes as wild-type mice) or the same mice endowed with an ability to express a green fluorescence protein (GFP) (hereinafter referred to sometimes as GFP-expressing mice) were euthanized manually by cervical spine dislocation under anesthesia with diethyl ether, and the whole body was antisepticised with 70 vol % of aqueous ethyl alcohol solution. The skin in the lower extremities below the lumbar region was removed with sharp-pointed tweezers and scissors previously sterilized by a process of steaming under high pressure. For preventing contamination with blood cell components due to bleeding as much as possible, the femoral artery exposed on the inguinal region was ligated with straight grasping forceps, and then the artery below the ligated portion within the visible region was exfoliated. Only muscles were removed carefully so as not to be contaminated with other blood vessels, nerves, tendons, ligaments and bone tissues, and the removed muscle tissues were rinsed in Hank' Balanced Salt Solution (manufactured by GIBCO) containing 2 mM of L-glutamine (manufactured by ICN Biomedicals) and 1 vol % of penicillin-streptomycin (manufactured by GIBCO) (hereinafter referred to as buffer 1) until blood components were sufficiently removed, and then the muscle tissues were stored in the fresh buffer 1. Then, the tissues in the buffer 1 were disrupted with sharp-pointed tweezers, while tissues other than the muscle were removed as much as possible. The disrupted muscle fragments were cut into thin fragments of about 1 mm³ or less with sterilized scissors until they became muddy. The muscle fragments together with the buffer 1 were recovered in a 50-ml conical tube and centrifuged once to remove a supernatant. Then, about 2 g of the muscle tissues were subjected to enzyme treatment by adding 15 ml of 0.4% collagenase type 2 (manufactured by Worthington Biochemical Corporation) previously kept at 37° C. and then shaking it for 45 minutes in a thermostat bath at 37° C. After the enzyme treatment, 20 ml of the buffer 1 was added per tube, then the sample was stirred well and centrifuged to remove a supernatant, and then 10 ml of DMEM (manufactured by GIBCO) culture medium containing 10 vol % of FBS (fetal bovine serum) (manufactured by Hyclone) and 1 vol % of penicillin-streptomycin was added to the sample to suspend the cells sufficiently, which were then filtered with a 100-μm cell strainer (manufactured by FALCON) and further filtered with a 40-μm cell strainer (manufactured by FALCON) to give mouse skeletal muscle tissue-derived cells.

### (2) Culture for Acquisition of Mouse Skeletal Muscle Tissue-Derived Stem Cell

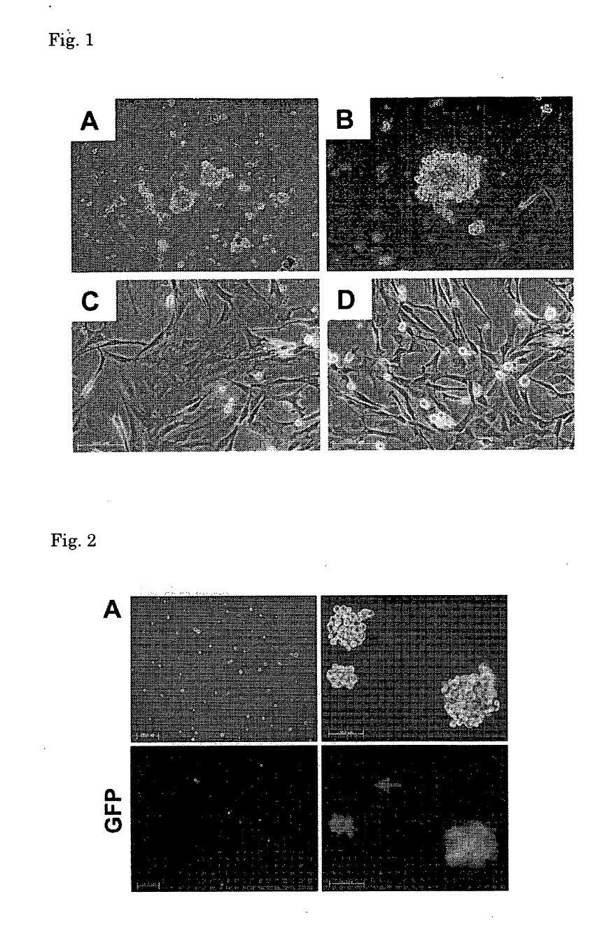

The skeletal muscle tissue-derived cells derived from the wild-type mice or the GFP-expressing mice, obtained in (1) above, were suspended in a serum-free medium [DMEM/F12 (manufactured by GIBCO) containing 2 vol % of a B27 supplement (manufactured by GIBCO), 1 vol % of an L-glutamine (200 mM)-penicillin (10000 units/ml)-streptomycin (10 mg/ml) solution (manufactured by SIGMA), 20 ng/ml of recombinant human basic FGF (manufactured by Promega) and 40 ng/ml of mouse EGF (manufactured by SIGMA)] (referred to hereinafter as “culture medium A”) and then counted. Using the culture medium A, the cells were cultured at a density of 2×10⁴ cells at the start of culture, at 37° C. under 5% CO₂ for 14 days in a non-coating 10-cm cell culture dish (manufactured by Corning, Inc.). By this culture, stem cell colonies floating in the culture medium (FIGS. 1A and B) were recognized, and simultaneously fibroblasts with a small nucleus extending in an elongated shape (FIG. 1C) and skeletal myoblasts spherically forming a population (FIG. 1D), both adhering to the bottom of the culture dish, were recognized.

### (3) Isolation of Colony of Mouse Skeletal Muscle Tissue-Derived Stem Cell and Culture of the Stem Cell

The cell groups (colonies) proliferating while floating in the culture medium, obtained in (2) above, were recovered through a micropipette. This sample was added to a fibronectin coating 24-well plate (Becton Dickinson) such that one cell group (colony) was put to each well, and then the cells were cultured again in a low-serum expansion medium [advanced DMEM/F12 (manufactured by GIBCO) containing 2 vol % of FBS, 1 vol % of an L-glutamine (200 mM)-penicillin (10000 units/ml)-streptomycin (10 mg/ml) solution (manufactured by SIGMA), 10 ng/ml of recombinant human basic FGF (manufactured by Promega), 20 ng/ml of mouse EGF (manufactured by SIGMA), and 10 ng ng/ml of mouse LIF (manufactured by CHEMICON)] (referred to hereinafter as “culture medium B”), whereby a monoclonal stem cell group was obtained.

For comparative study, the cells adhering to the 10-cm fibronectin coating dish (referred to hereinafter as “adherent cells”) after culture in (2) above were also recovered by enzyme treatment.

### (4) Culture for Acquisition of Mouse Skeletal Muscle Tissue-Derived Stem Cell and Isolation of Colony of the Stem Cell

The wild-type mouse skeletal muscle tissue-derived cells obtained in (1) above and the GFP-expressing mouse skeletal muscle tissue-derived cells obtained in (1) above were mixed at a ratio of 1:1 and cultured in a non-coating 10-cm cell culture dish under the same conditions as in (2) above. The results of observation of floating cells or colonies before and after culture are shown in FIG. 2. In FIG. 2, the upper left photograph is a phase-contrast micrograph of floating cells at the start of culture on a non-coating 10-cm cell culture dish; the lower left photograph is a fluorescence micrograph of the same visual field as in the upper left photograph; the upper right photograph is a phase-contrast micrograph of floating colonies after culture on a non-coating dish for 14 days; and the lower right photograph is a fluorescence micrograph of the same visual field as in the upper left photograph. The colony observed at the part indicated by the arrow in the upper left photograph in FIG. 2 was not observed in the lower left photograph, and thus this colony can be seen to be a colony formed by proliferation of the wild-type mouse skeletal muscle tissue-derived cells. On the other hand, the colonies observed outside the part indicated by the arrow in the upper left photograph in FIG. 2 are also observed in the lower left photograph, and thus these colonies are colonies formed by proliferation of the GFP-expressing mouse-derived cells or are cell masses formed by aggregation of the wild-type mouse-derived cells and the GFP-expressing mouse-derived cells. Accordingly, the colony indicated by the arrow in the upper left photograph in FIG. 2 is collected, whereby a colony formed from a single cell can be accurately isolated without erroneously collecting a pseudo-stem cell colony that is a cell aggregate.

### (5) Confirmation of Characteristics of Mouse Skeletal Muscle Tissue-Derived Stem Cell

### <Expression of BrdU>

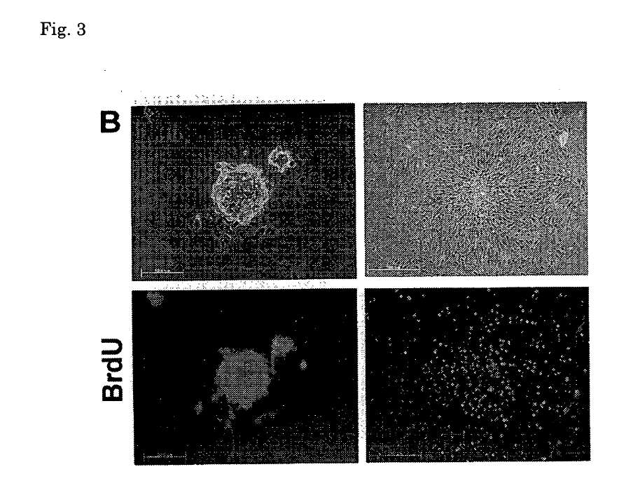

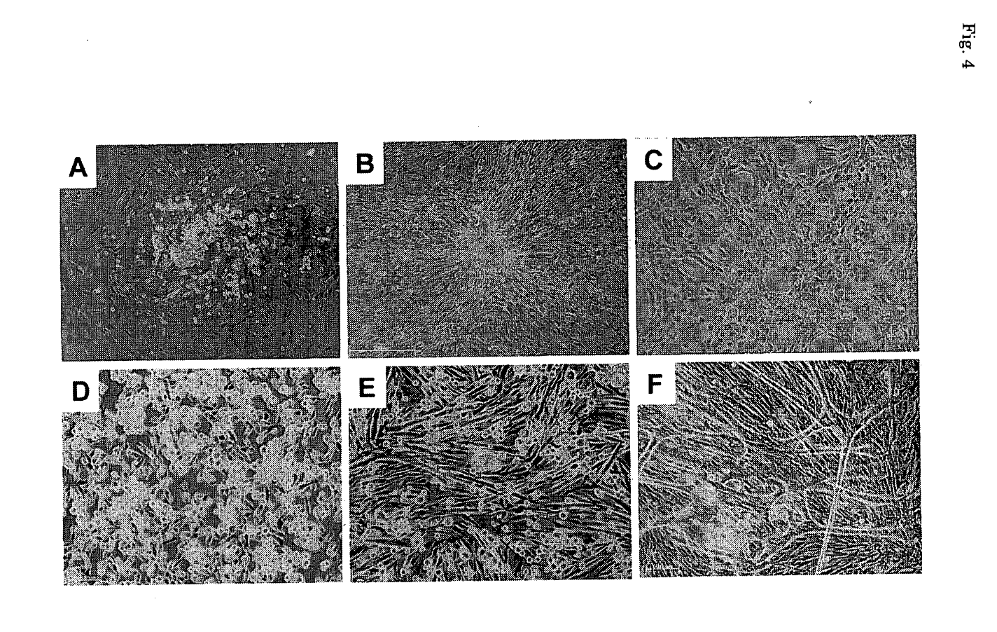

Bromodeoxyuridine (BrdU) in the isolated stem cells isolated in (3) above was stained to confirm whether BrdU had been incorporated into the cells or not. Whether BrdU had been incorporated into marginal cells generated by culturing and proliferating, in the culture medium B at 37° C. for 5 days, the stem cells isolated in (3) above was also confirmed in the same way. The results are shown in FIG. 3. In FIG. 3, the lower left photograph shows the result (fluorescence micrograph) of observation of expression of BrdU in the stem cells isolated in (3) above; the upper left photograph is a phase-contrast micrograph of the same visual field as in the lower left photograph; the lower right photograph shows the result (fluorescence micrograph) of observation of expression of BrdU in the marginal cells generated by culturing and proliferating for 5 days the stem cells isolated in (3) above; and the upper right photograph is a phase-contrast micrograph of the same visual field as in the lower right photograph. As can be seen from FIG. 3, the stem cells isolated in (3) above were BrdU-positive and confirmed to undergo active cell division. The marginal cells generated by culturing and proliferating for 5 days the stem cells isolated in (3) above were also confirmed to be BrdU-positive.

### <Proliferation Characteristics>

The stem cells isolated in (3) above when cultured in the culture medium B in a 10-cm fibronectin coating dish (manufactured by Becton Dickinson) at 37° C. under 5% CO₂ for 7 days were confirmed to proliferate with time (see FIGS. 4A and B). In FIG. 4, A is a micrograph of the cells on day 3 of culture, and B is a micrograph of the cells on day 7 of culture. When the stem cells isolated in (3) above were cultured in the culture medium B, a high-purity cell population of the stem cells not contaminated with other type of cells was able to be obtained.

When the stem cells isolated in (3) above were passaged every 6 days, their proliferation ability was maintained even after culture with a passage number of 3 (see FIG. 4C). In FIG. 4, C is a micrograph of the cells after culture with a passage number of 3.

On the other hand, the adherent cells isolated in (3) above, when cultured in the culture medium B in a 10-cm fibronectin coating dish at 37° C. under 5% CO₂ for 7 to 10 days, were confirmed to proliferate with time in a state adhering to the dish, to form myotubes and differentiate into skeletal muscle cells (see FIG. 4D to F). In FIG. 4, D shows a micrograph showing the proliferated adherent cells; E, the state of formation of myotubes from the adherent cells; and F, the state of differentiation of the adherent cells into skeletal muscle cells. It was thus confirmed that the adherent cells are skeletal myoblasts present in the skeletal muscle tissue.

### <Cell Surface Antigens and Markers>

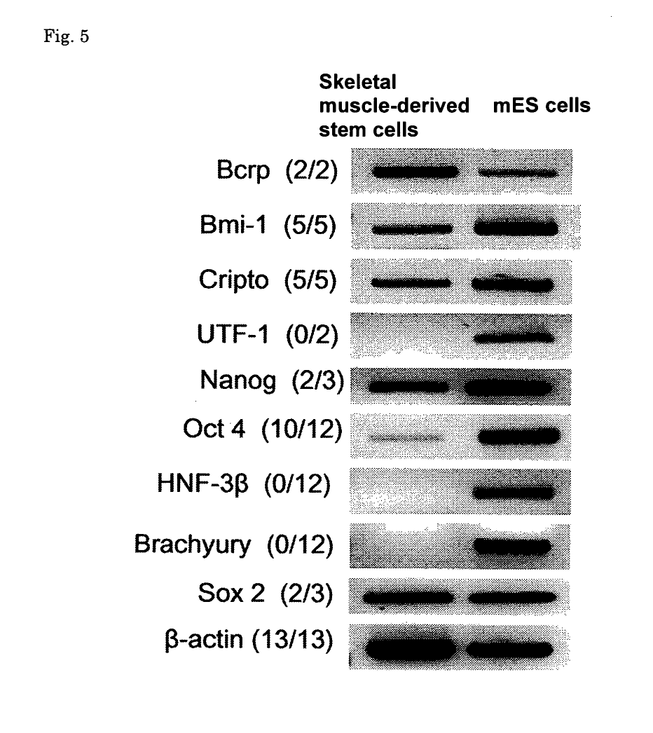

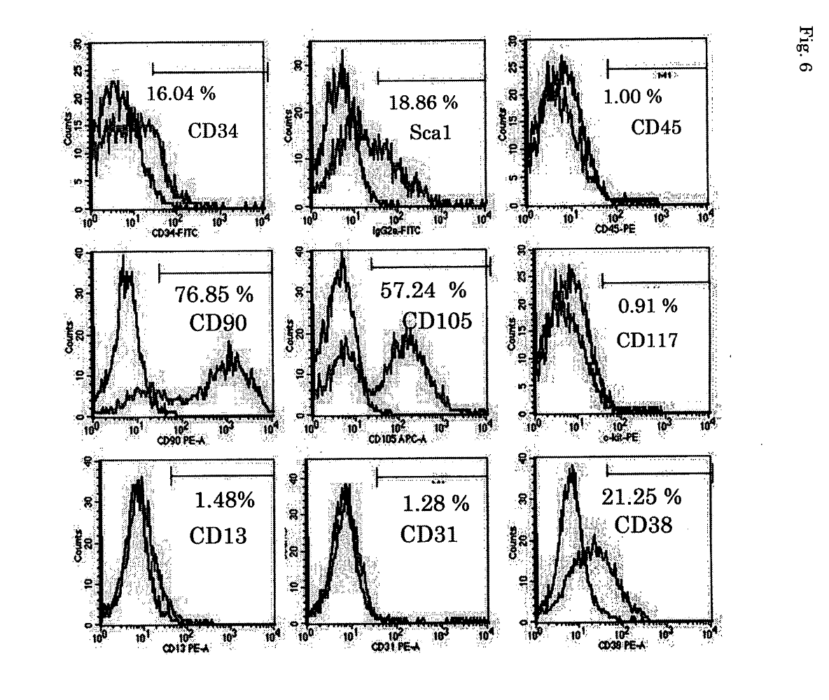

The stem cells isolated in (3) above were analyzed by PCR for their expression of ES cell markers (Bcrp, Bmi-1, Cripto, UTF-1, Nanog, Oct-4, HNF-3β, Brachyury, Sox2 and β-actin). The results are shown in FIG. 5. The results indicted that the stem cells isolated in (3) above, similar to ES cells, strongly express Bcrp, Bmi-1, Cripto, Nanog, Oct-4 and Sox 2.

Further, the stem cells isolated in (3) above were analyzed by FACS for various cell surface antigens (CD34, Sca-1, CD45, CD90, CD105, CD117, CD13, CD31 and CD38). The results are shown in FIG. 6. From the result, it was confirmed that CD90 and CD105 are strongly positive; CD34, Sca-1, CD13 and CD38 are weakly positive; and CD45, CD117 and CD31 are negative.

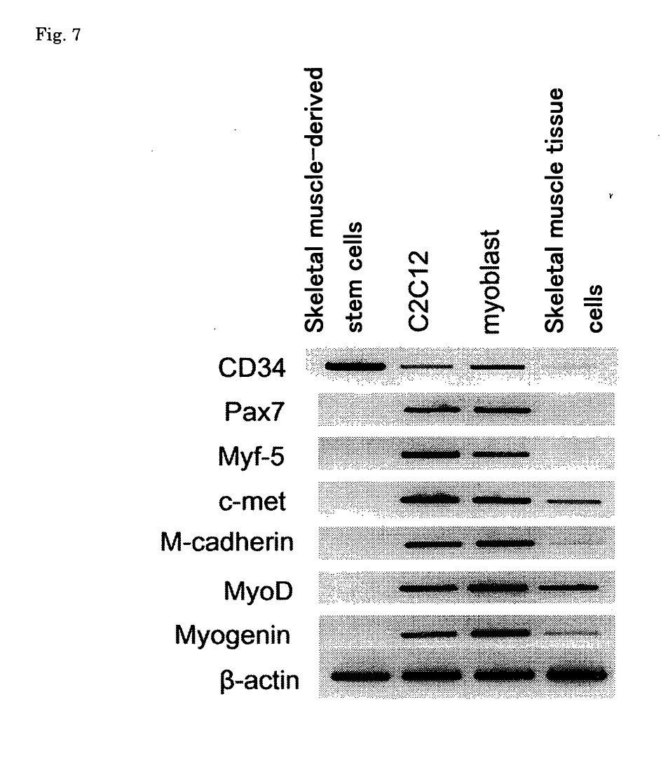

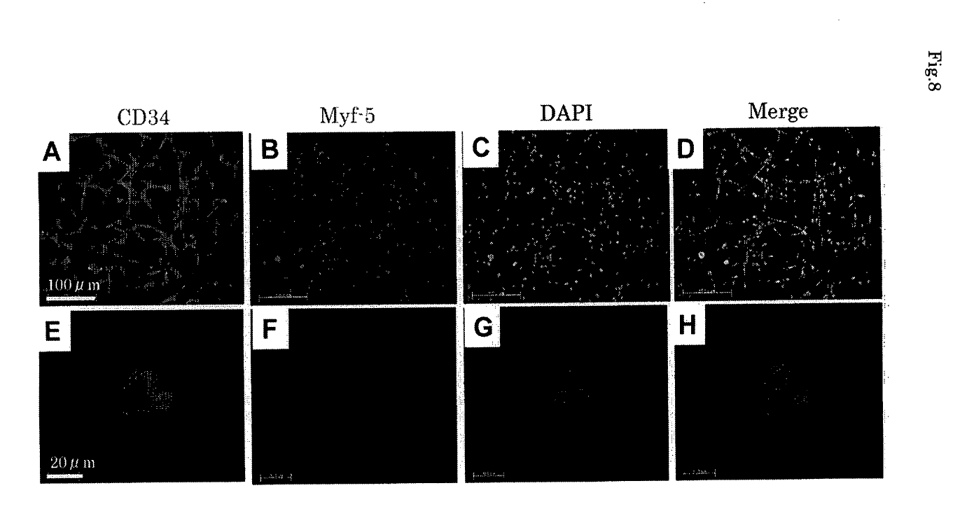

Further, the stem cells isolated in (3) above were analyzed by PCR for their expression of myoblast C2C12 and skeletal myoblast markers (Pax-7, Myf-5, c-met, M-cadherin, MyoD and Myogenin). The results are shown in FIG. 7. From the result it was conformed that the stem cells isolated in (3) above do not express the markers recognized in myoblast C2C12 or skeletal myoblast.

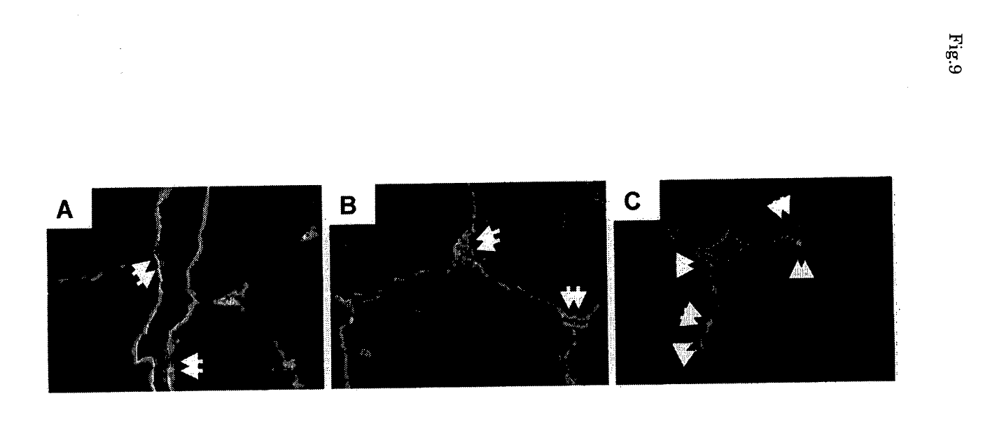

In the stem cells isolated in (3) above, CD34 was stained red with Alex Fluor 555 (manufactured by Molecular Probes); Myf-5 was stained green with Alex Fluor 488 (manufactured by Molecular Probes); and intracellular nuclei were stained blue with DAPI (4′,6-diamino-2-phenylindole). The adherent cells collected in (3) were also stained in a similar manner. The results are shown in FIG. 8. In FIG. 8, A to D show the results of staining of the adherent cells, and E to F show the results of staining of the stem cells isolated in (3) above. From the results, it was also confirmed that the stem cells isolated in (3) are CD34-positive, and do not express Myf-5 that is a transcription factor of the skeletal muscle cell. A part of the adherent cells were confirmed to be CD34- and Myf-5-positive to show characteristics of a satellite cell.

### Example 2 Investigation of the Site where the Mouse Skeletal Muscle Tissue-Derived Stem Cells Are Localized

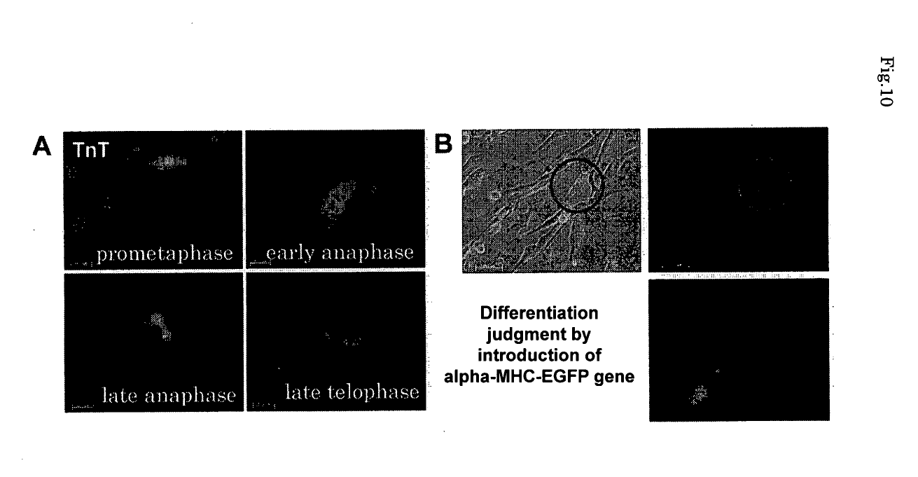

From 6- to 8-week-old female C57Bl/6J mice (available from Shimizu Laboratory Supplies Co., Ltd.), skeletal muscle fragments were collected in a usual manner, and the skeletal muscle fragments were used as the sample, wherein laminine was stained green with Alex Fluor 488 (manufactured by Molecular Probes); intracellular nuclei were stained blue with DAPI; and CD34 was stained red with Alex Fluor 555 (manufactured by Molecular Probes). The sample thus stained was observed under a microscope to confirm whether the cells in the cell basement membrane and interstitial tissue were stained or not. The results are shown in FIGS. 9A and B. In FIG. 9, A is a micrograph of the cell basement membrane, and B is a micrograph of the interstitial tissue. From the results, it was confirmed that the skeletal myoblasts are present under the cell basement membrane (see FIG. 9A), and the stem cells are localized in the interstitial tissue (see FIG. 9B).

Similarly, the skeletal muscle fragments were used as the sample, wherein laminine was stained red with Alex Fluor 555 (manufactured by Molecular Probes); intracellular nuclei were stained blue with DAPI; and Myf-5 was stained green with Alex Fluor 488 (manufactured by Molecular Probes), and the sample was observed under a microscope to confirm whether the cells in the cell basement membrane and interstitial tissue were stained or not. The results are shown in FIG. 9C. From the results, it was confirmed that the Myf-5-positive skeletal myoblasts are present under the cell basement membrane (see the part indicated by yellow arrow in FIG. 9C), and the Myf-5-negative stem cells are present in the interstitial tissue (see the part indicated by white arrow in FIG. 9C).

### Example 3 Induction of Differentiation of Mouse Skeletal Muscle Tissue-Derived Stem Cells into Myocardial Cells

### Confirmation of Differentiation into Myocardial Cells by Morphological Observation and by Confirmation of GFP Expression

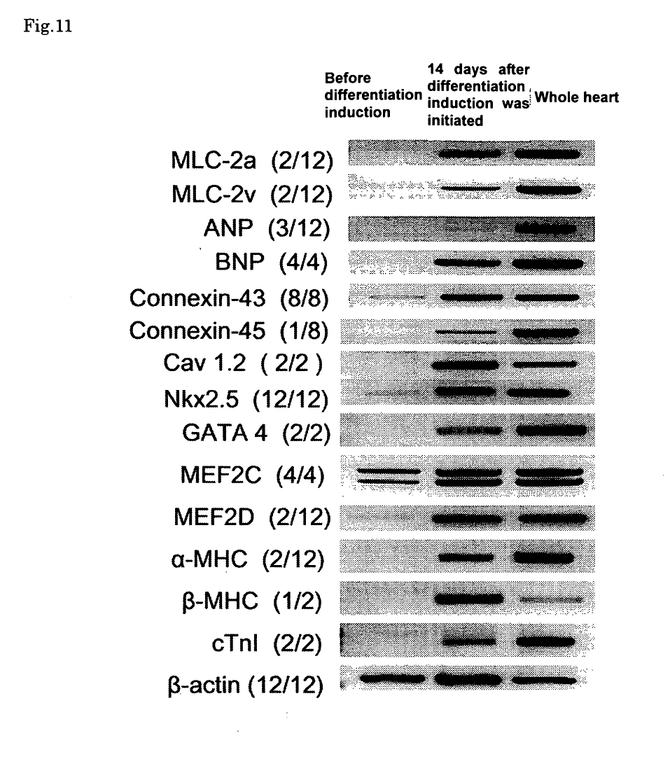

The stem cells obtained in (3) above were cultured in the culture medium B until the cells became subconfluent, and after the culture medium was exchanged with a culture medium for differentiation induction [MEM culture medium (manufactured by GIBCO); 10 vol % of FBS, 1 vol % of penicillin (10000 units/ml)-streptomycin (10 mg/ml), 1 vol % of insulin-transerrin-serenium-X (manufactured by GIBCO) and 1×10⁻⁸ M of dexamethasone (manufactured by SIGMA)], the cells were further cultured at 37° C. under 5% CO₂ for 2 to 3 weeks. From about 5 days after the culture medium was exchanged, the presence of spontaneously contracting cells was observed. From the results of observation of morphological characteristics of the cells after culture and results of staining of the cells with DAPI and cardiac-specific troponin-I, it was confirmed that the stem cells were differentiated into myocardial cells (see FIG. 10A). Because it was confirmed that the stem cells were differentiated into myocardial cells from prometaphase to late telophase (see FIG. 10A), it was confirmed that myocardial cells into which the stem cells were differentiated have an ability to proliferate through cell division.

Further, the stem cells proliferated in (3) above were cultured at 37° C. under 5% CO₂ for 2 to 3 weeks in the above-mentioned culture medium for differentiation induction. From about 5 days after differentiation induction was initiated, the presence of spontaneously contracting cells was observed. Ten days after differentiation induction was initiated, a GFP gene under the control of an alpha-myocardial heavy chain promoter was introduced in a usual manner into the cells, and the result of microscopic observation of the state of the cells 3 days thereafter is shown in FIG. 10B. In FIG. 10B, the upper left photograph is an image observed under a phase-contrast microscope; the upper right photograph is an image observed in the same visual field as in the upper left photograph under a fluorescence microscope; and the lower right photograph is an enlarged image of the circled part in the upper right photograph. As a result, a green color indicative of expression of GFP was observed, and thus the differentiation of the stem cells into myocardial cells was supported.

<Confirmation of Differentiation into Myocardial Cells by Confirmation of Markers>

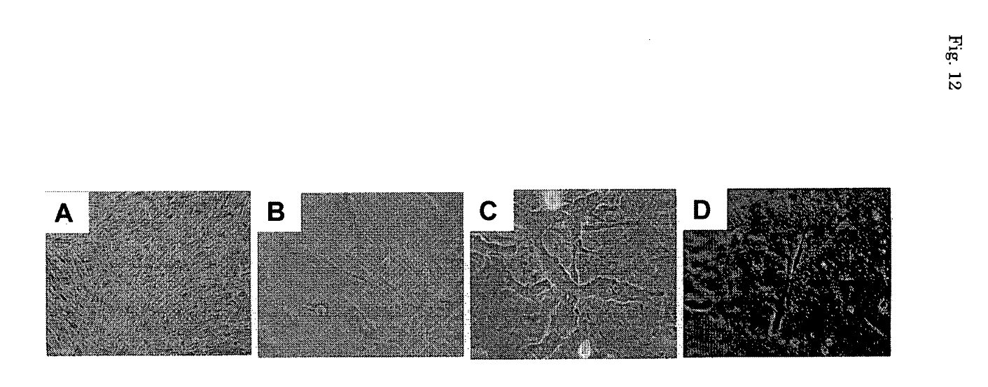

The stem cells obtained in (3) above, and cells into which the stem cells were induced to differentiate by culture at 37° C. under 5% CO₂ for 14 days in the culture medium for differentiation induction, were analyzed by PCR for expression of various markers (various myocardial transcription factors, structural proteins, myocardium-binding proteins, and calcium ion channels shown in FIG. 11) recognized in myocardial cells. The results are shown in FIG. 11. As a positive control, the whole heart (positive control) was used. As a result, it was confirmed that 14 days after differentiation induction, the stem cells were differentiated into myocardial cells.

<Confirmation of Differentiation into Myocardial Cells on the Basis of Morphological Characteristics>

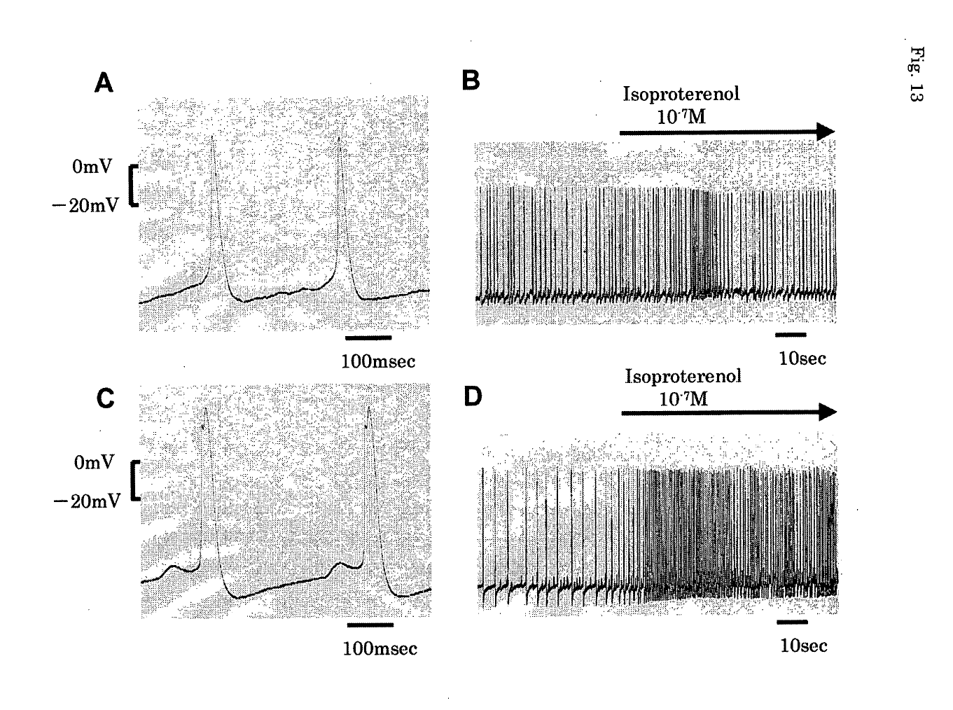

When the differentiation of skeletal myoblasts is induced in a usual manner, pulsatile myotubes proliferated and differentiated in a sheet shape are formed (see FIG. 12A). Further, skeletal myoblasts remaining undifferentiated may form a multinucleate cell state to show rhythmical beating (see FIG. 12B). As just described, the cells though not differentiated into myocardial cells may show pulsatility and exhibit characteristics similar to those of myocardial cells.

On the other hand, when differentiation of the stem cells obtained in (3) above is induced under the conditions described above, differentiation into pulsatile myocardial cells in a mononuclear or binuclear state (see FIG. 12C), particularly differentiation into regularly beating mononuclear myocardial cells having a lower beating rate than that of skeletal myoblasts (see FIG. 12D), is recognized. From these results, it was also confirmed the stem cells are differentiated truly into myocardial cells.

<Confirmation of Differentiation into Myocardial Cells by Electrophysiological Examination>

Differentiation of the stem cells obtained in (3) above was induced under the conditions described above, and the resulting myocardial cells were tested for their beating by the following method and examined electrophysiologically. For comparison, skeletal muscle cells were also similarly electrophysiologically examined.

Specifically, the stem cells obtained in (3) above were cultured at 37° C. under 5% CO₂ in the above-mentioned culture medium for differentiation induction on a collagen-coated cover glass (3 mm×7 mm, thickness 0.17 to 0.25 mm, manufactured by MATSUNAMI GLASS IND, LTD.), and 7 to 10 days after differentiation induction was initiated, the membrane potential was measured. The conditions for measurement of the membrane potential are as follows: The cover glass having the above cultured cells adhering thereto was introduced into a recording chamber perfused with a Tyrode solution (containing 140 mM of NaCl, 0.33 mM of NaHPO₄, 5.4 mM of KCl, 1.8 mM of CaCl₂, 0.5 mM of MgCl₂, 5.5 mM of glucose and 5 mM of HEPES; adjusted to pH 7.4 with NaOH), then borosilicate glass capillaries regulated at a resistance of 2 to 3 MΩ filled with a pipette solution (containing 120 mM of KCl, 1 mM of MgCl₂, 10 mM of EGTA, 10 mM of HEPES, and 3 mM of MgATP; adjusted to pH 7.2 with KOH), and the action potential was recorded (whole cell recording) with an amplifier (Axopatch 200A, manufactured by Axon Instruments).

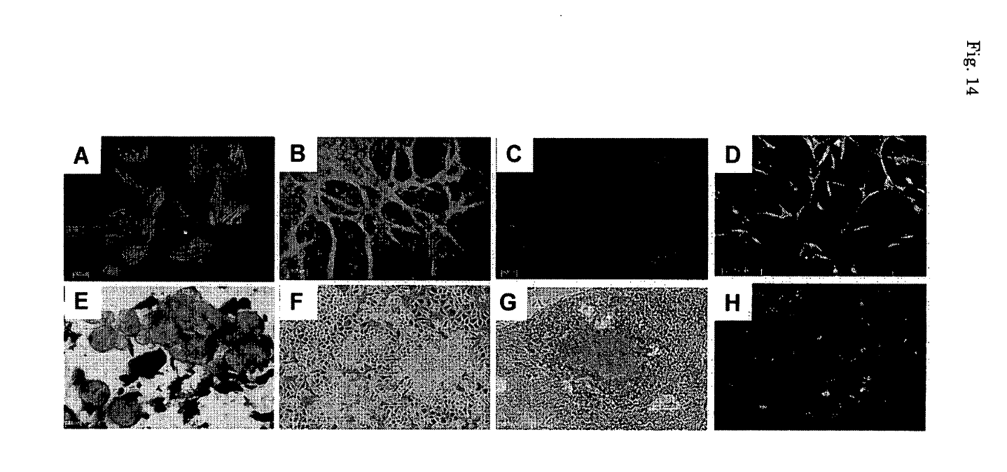

The results thus obtained are shown in FIG. 13. In FIG. 13, A shows an amplitude pattern of action potential of the skeletal muscle cells; B shows an amplitude pattern of action potential of the skeletal muscle cells before and after addition of isoproterenol; C shows an amplitude pattern of action potential of myocardial cells formed through differentiation induction from the stem cells; and D shows an amplitude pattern of action potential, before and after addition of isoproterenol, of myocardial cells formed through differentiation induction from the stem cells. As can be seen from FIGS. 13A and C, the myocardial cells into which the stem cells were induced to differentiate showed broader action potential with notch than that of skeletal muscle cells. As can be clearly seen from FIGS. 13B and D, the myocardial cells into which the stem cells were induced to differentiate showed a significant increase in beating rate upon addition of isoproterenol, but the skeletal muscle cells did not show a change in beating rate before and after addition of isoproterenol. From these results, it was also confirmed that the myocardial cells into which the stem cells were induced to differentiate show beating behavior inherent in myocardial cells, and this beating behavior is different from that of the skeletal muscle.

### Example 4 Induction of Differentiation of the Mouse Skeletal Muscle Tissue-Derived Stem Cells into Other Cells

The stem cells obtained in (3) above were cultured in the culture medium B until the cells became subconfluent, then the culture medium was exchanged with an MEM culture medium supplemented with various inducers, and the stem cells were cultured at 37° C. under 5% CO₂ for additional 2 to 3 weeks thereby inducing differentiation into various types of cells. The inducers used for induction of differentiation into various types of cells and the concentrations thereof are as shown in Table 1.

TABLE 1

| Differentiation-Induced | |
|---|---|
| Cells | Inducers and Their Concentrations |
| Vascular smooth muscle | 10 ng/ml platelet-derived growth factor |
| cell | (PDGF-BB) |
| Endothelial cell | 10 ng/ml vascular endothelial growth factor |
| | (VEGF) |
| Glia cell | 5 mM β-mercaptoethanol |
| Neuronal cell | 10 ng/ml |
| Fat cell | 1 × ITS-A, 1 × 10⁻⁸ M dexamethasone, and |
| | 0.5 mM IBMX (3-Isobutyl-1-methylxanthine) |
| Epithelial cell | 1 × 10⁻⁸ M dexamethasone |
| Bone cell | 10 ng/ml TGF (Transforming growth factor), |
| | 50 mM ascorbic acid-2-phosphate |
| Skeletal muscle cell | 5 vol % FBS (fetal bovine serum) |

When the cells were cultured in the various culture mediums and then observed, it was confirmed that the stem cells obtained in (3) were differentiated into vascular smooth muscle cells (FIG. 14A), endothelial cells (FIG. 14B), glia cells (FIG. 14C), neuronal cells (FIG. 14D), fat cells (FIG. 14E), epithelial cells (FIG. 14F), bone cells (FIG. 14G), and skeletal muscle cells (FIG. 14H). Differentiation into these cells was confirmed on the basis of the morphological characteristics of the cells and results of staining of the various types of cells. The stem cells were judged to be differentiated into vascular smooth muscle cells by staining with α-SMC, endothelial cells by staining with CD31, glia cells by staining with GAFP, neuronal cells by staining with NF2000, fat cells by staining with oil-red, bone cells by staining with Alizarin red, and skeletal muscle cell by staining with fast skeletal-MHC. From these results, the stems cells obtained in (3) above were found to be pluripotent stem cells capable of differentiation into multiple organs.

### Example 5 Transplantation of Mouse-Derived Stem Cells

By the same method as in Example 1, skeletal muscle tissue-derived stem cells were obtained from LacZ-overexpressing mice having cells systemically expressing LacZ, and then cultured and proliferated. The LacZ-expressing stem cells (about 1×10⁶ cells) thus obtained were suspended in 15 μl of PBS(−) (manufactured by GIBCO) and transplanted via BD Ultra Fine II Lancet (manufactured by Becton Dickinson) into infarcted cardiac muscles generated in 10- to 12-week-old C57Bl/6J mice (available from Shimizu Laboratory Supplies Co., Ltd.). Twenty-one days after the stem cells were transplanted, the heart was excised from each mouse. In the heart muscle of the excised heart, LacZ was stained green, while intracellular nuclei were stained blue with DAPI, the myocardial cells were stained red with cardiac-specific troponin-I, the endothelial cells were stained red with CD31, and the vascular smooth muscle cells were stained red with α-smooth muscle-MHC.

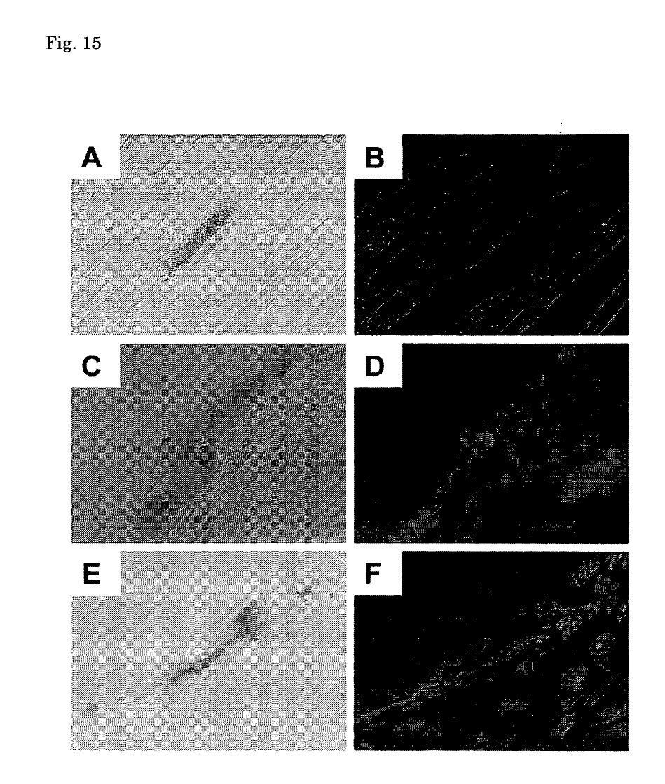

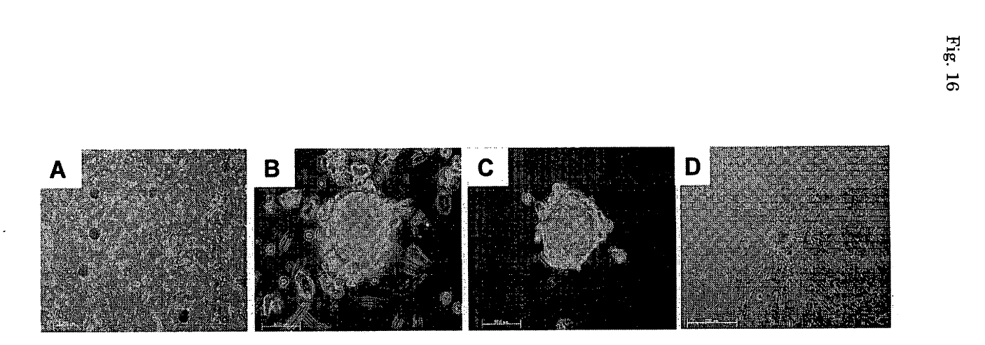

The results thus obtained are shown in FIG. 15. In FIGS. 15, A, C and E show the results of staining of LacZ; B shows the result of staining using cardiac-specific troponin-I in the same visual field as in A; D shows the result of staining using CD31 in the same visual field as in C; and F shows the result of staining using α-smooth muscle-MHC in the same visual field as in E. From these results, it was revealed that the transplanted skeletal muscle-derived stem cells were engrafted in the host cardiac muscle and differentiated into myocardial cells, endothelial cells and vascular smooth muscle cells, thus contributing to repair of the heart.

### Example 6 Acquisition of Human-Derived Stem Cell

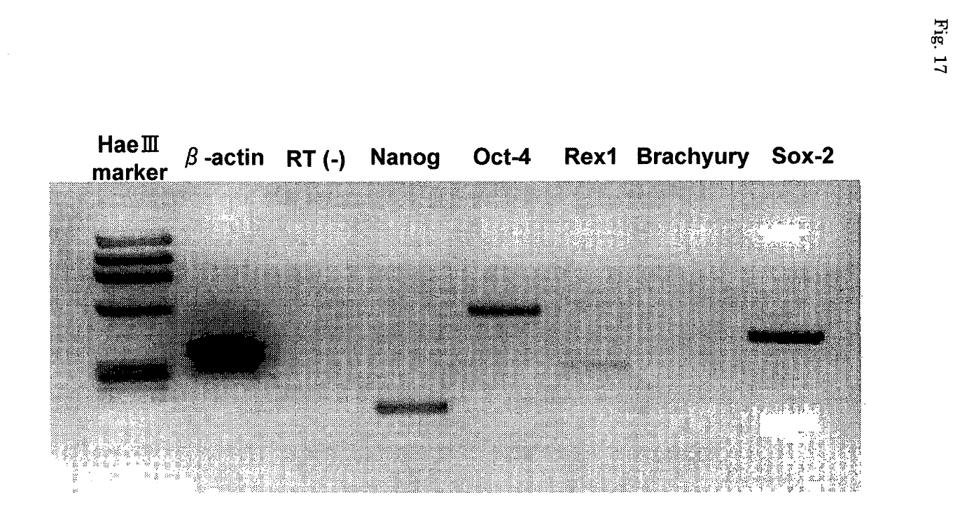

When human skeletal muscle tissue-derived cells in skeletal muscle fragments collected from a human were cultured according to “(1) Acquisition of Mouse Skeletal Muscle Tissue-Derived Cell” and “(2) Culture for Acquisition of Mouse Skeletal Muscle Tissue-Derived Stem Cell” described in Example 1, a cell group (colony) floating in the culture medium and showing a proliferation ability, together with proliferated cells adhering to a dish, was recognized (see FIGS. 16 A and B).

The colony formed from a single stem cell was mechanically isolated and cultured according to “(3) Isolation of Colony of Mouse Skeletal Muscle Tissue-Derived Stem Cell and Culture of the Stem Cell” described in Example 1, thereby successfully isolating and culturing a human skeletal muscle tissue-derived stem cell. A microgram of the stem cell colony just after isolation is shown in FIG. 16C, and a microgram of the stem cell colony proliferated in a concentric form after 5 days of culture is shown in FIG. 16D.

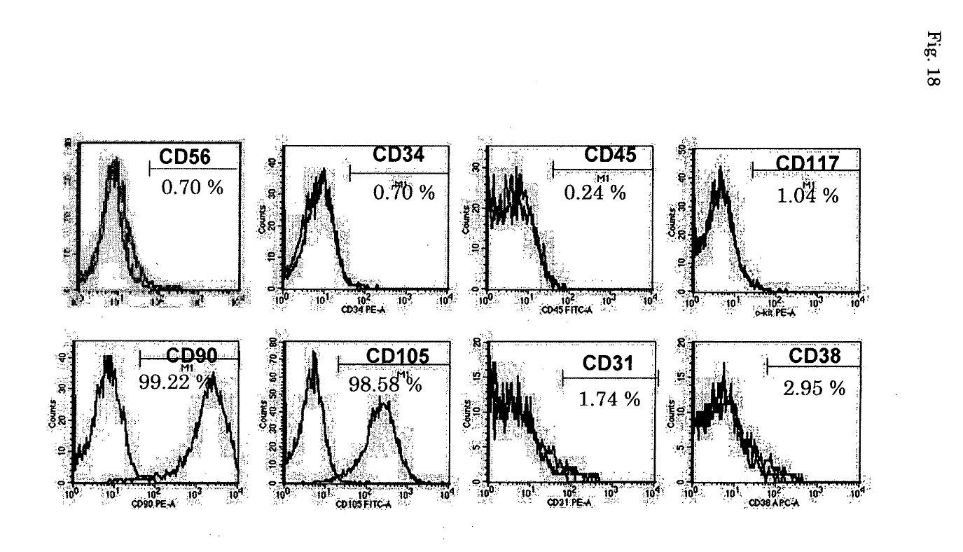

The human skeletal muscle tissue-derived stem cells obtained above were analyzed by RT-PCR for their expression of ES cell markers (Nanog, Oct-4, Rex1, Brachyury and Sox 2). The results are shown in FIG. 17. As a result, it was confirmed that the isolated stem cells described above express Nanog, Oct-4, Rex1 and Sox 2 similarly to the ES cell.

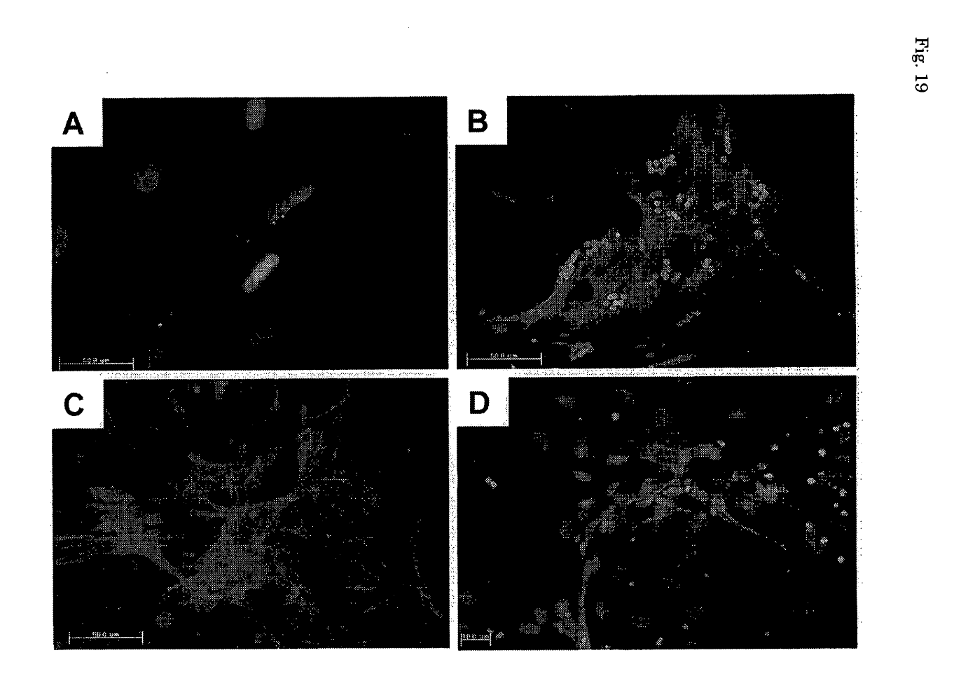

The human skeletal muscle tissue-derived stem cells obtained above were analyzed by FACS for various cell surface antigens (CD56, CD34, CD45, CD117, CD90, CD105, CD31 and CD38). The results are shown in FIG. 18. CD56 is a surface antigen recognizing 100% human skeletal myoblasts. The human skeletal muscle tissue-derived stem cells were almost CD56-negative. The results of the human skeletal muscle tissue-derived stem cells were significantly different from the mouse-derived stem cells in that the human cells are CD34-negative and CD38-weakly positive. On the other hand, the human- and mouse-derived stem cells agreed with each other in that both were CD105- and CD90-positive. Sca-1 was not examined because it does not occur in human-derived cells.

The stem cells obtained above were confirmed to be c-met-negative, Pax-7-negative, Myf-5-negative, MyoD-negative, Myogenin-negative and M-cadherin-negative as determined by measuring expression of the markers.

### Example 7 Induction of Differentiation of the Human Skeletal Muscle Tissue-Derived Stem Cells into Various Types of Cells

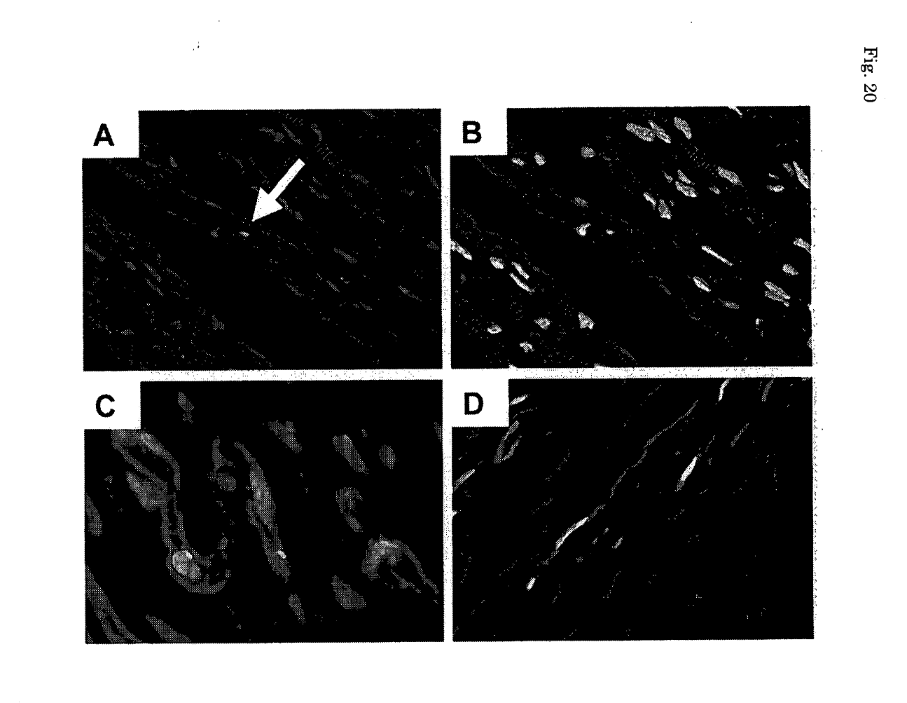

By the same method as in Example 4, the human skeletal muscle tissue-derived stem cells obtained in Example 6 were induced to differentiate into various types of cells. It was thereby confirmed that the stem cells were differentiated into myocardial cells (see FIG. 19A), into skeletal muscle cells (see FIG. 19B), into smooth muscle cells (see FIG. 19C), and into endothelial cells (see FIG. 19D). Differentiation into these cells was confirmed on the basis of the morphological characteristics of the cells and results of staining of the various types of cells. Cell staining involved staining of intracellular nuclei with DAP plus staining with cardiac-specific troponin-I for myocardial cells, staining with fast muscle-type skeletal myosin heavy chain for skeletal muscle cells, smooth muscle myosin heavy chain for smooth muscle cells, and CD31 for endothelial cells. From these results, the human skeletal muscle tissue-derived stem cells obtained in Example 6 were confirmed to be pluripotent stem cells capable of differentiation into multiple organs.

### Example 8 Transplantation of the Human Skeletal Muscle Tissue-Derived Stem Cells

The human skeletal muscle tissue-derived stem cells (about 1×10⁶ cells) obtained in Example 6 were suspended in 15 μl of PBS(−) (manufactured by GIBCO) and transplanted via BD Ultra Fine II Lancet (manufactured by Becton Dickinson) into infarcted cardiac muscles generated in 10- to 12-week-old NOD/SCID mice (purchased from Jackson Laboratory). Twenty-one days after the stem cells were transplanted, the heart was excised from each mouse. In the heart muscle of the excised heart, the intracellular nuclei were stained with DAPI (4′,6-diamino-2-phenylindole); the myocardial cells were stained red with cardiac-specific troponin-I; the smooth muscle cells were stained red with alpha-smooth muscle myosin heavy chain; the endothelial cells were stained red with CD31; and the human-derived nuclei were stained green with a mouse anti-human nuclei monoclonal antibody.

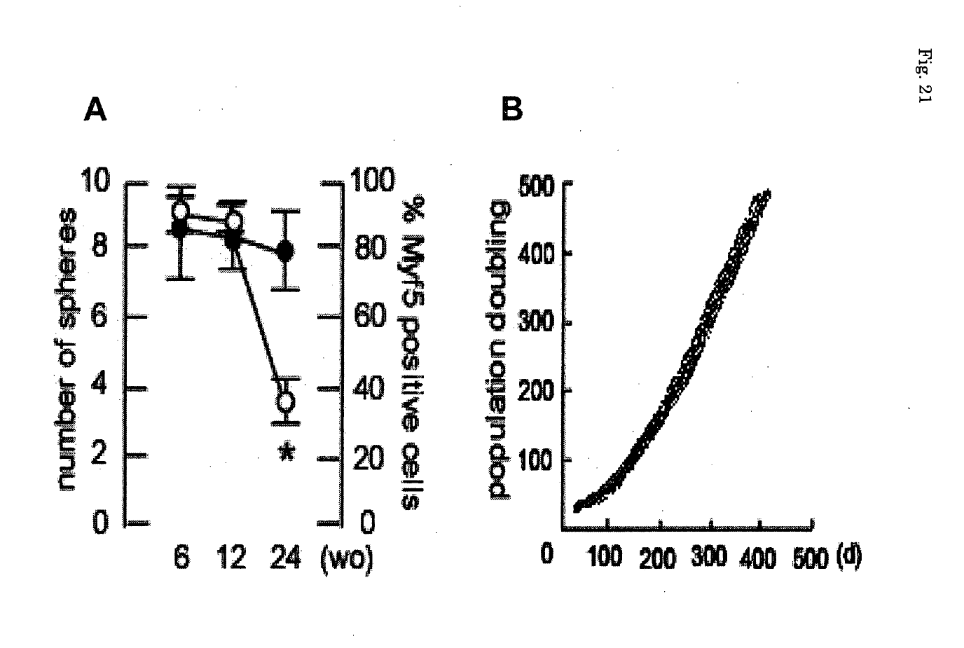

The results are shown in FIG. 20. In FIG. 20, A shows the results of staining myocardial troponin-I-positive myocardial cells red and staining human-derived nuclei green; B shows the results of staining myocardial troponin-I-positive myocardial cells red and staining all nuclei white with DAPI, in the same visual field as in A; C shows the results of staining alpha-smooth muscle myosin heavy chain-positive smooth muscle cells red and staining human-derived nuclei green; and D shows the results of staining CD31-positive endothelial cells red and staining human-derived nuclei green. From these results, it was confirmed that the human skeletal muscle-derived stem cells were engrafted in the host cardiac muscle and differentiated into myocardial cells (see the part indicated by white arrow in FIG. 20A). It was also confirmed that the human skeletal muscle-derived stem cells were engrafted in the host cardiac muscle and differentiated into smooth muscle cells and endothelial cells (see FIGS. 20C and D). From these results, it was revealed that the transplanted skeletal muscle-derived stem cells were engrafted in the host cardiac muscle and differentiated into the various types of myocardial cells, thus contributing to repair of the heart.

### Example 9 Investigation of Long-Term Culture of Mouse Skeletal Muscle-Derived Stem Cells

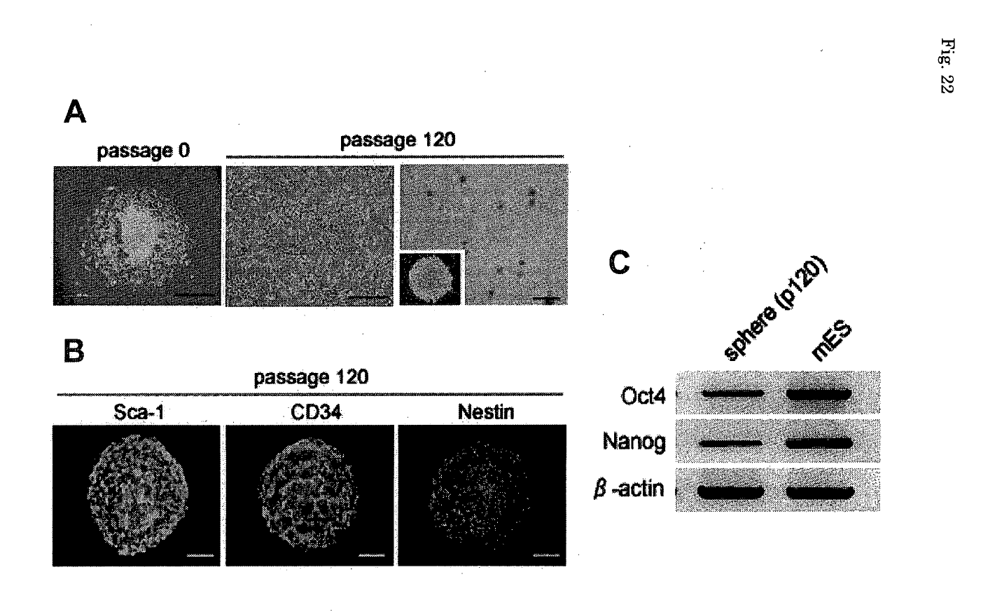

From 6-, 12- and 24-week-old female C57Bl/6J mice (available from Shimizu Laboratory Supplies Co., Ltd.), mouse skeletal muscle-derived stem cells were obtained in the same manner as in (1) to (3) in Example 1. The successfully obtained mouse skeletal muscle-derived stem cell colonies and satellite cells adhering to a fibronectin coating dish were counted respectively. The results are shown in FIG. 21A. The results indicated that the Myf5-positive satellite cells (white circles in FIG. 21A) are decreased as the age of mice in week is increased. On the other hand, the content of skeletal muscle-derived stem cells in the tissues (black circles in FIG. 21A) did not change and remained constant regardless of an increase in the age of mice in week.

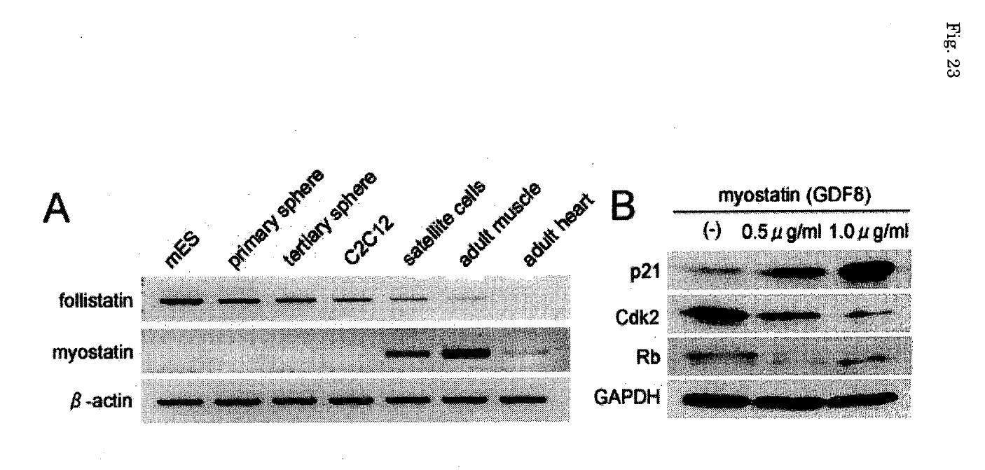

When the skeletal muscle-derived stem cells obtained from 6-week-old female C57Bl/6J mice were passaged in the culture medium B, it was confirmed that the stem cells can be cultured for 400 days with a passage number of 120 (see FIG. 21B). The form of the cells observed during this passage culture is shown in FIG. 22. In FIG. 22A, the left photograph shows the state of initially formed stem cell colonies (passage number, 0); the middle photograph shows the state of the cells which were subjected to adhesion culture with a passage number of 120; and the right photograph shows the state of the stem cells which while forming colonies, were amplified after culture with a passage number of 120 by exchanging the culture medium with a serum-free culture medium.

Whether the skeletal muscle-derived stem cell colonies cultured with a passage number of 120 express stem cell markers Sca-1, CD34 and nestin was confirmed by cell staining. As a result, it was able to be confirmed that the stem cell colonies cultured with a passage number of 120 present any of the stem cell markers mentioned above and is maintained in an undifferentiated state (see FIG. 22B). The stem cell colonies cultured with a passage number of 120 were analyzed by PCR for their expression of ES cell markers Nanog and Oct-4. From these results, it was also able to be confirmed that the stem cell colonies cultured with a passage number of 120 express Nanog and Oct-4 (see FIG. 22C).

### Example 10 Investigation of the Influence of Myostatin on Culture of the Skeletal Muscle-Derived Stem Cells

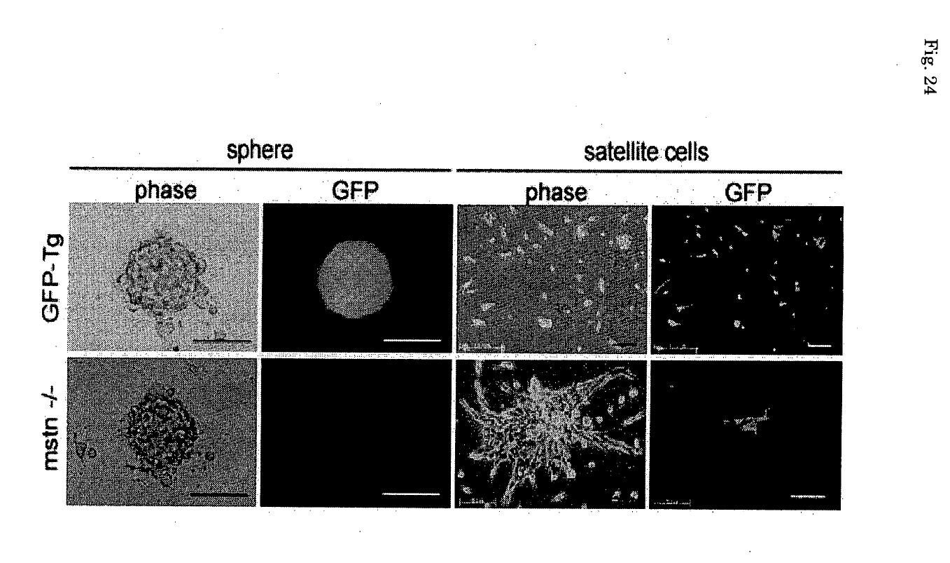

ES cells, the initial (passage number 0; primary sphere) skeletal muscle-derived stem cells obtained in Example 9, the skeletal muscle-derived stem cells (passage number 3; tertiary sphere) obtained in Example 9, mouse myoblasts (C2C12), satellite cells adhering to a fibronectin coating dish in the process for preparing the mouse skeletal muscle-derived stem cells in Example 9, adult human muscle-derived adult cells, and adult heart muscle-derived adult cells were analyzed respectively by PCR for their expression of follistatin and its antagonistic ligand myostatin. The results are shown in FIG. 23A. From these results, it was revealed that follistatin is strongly expressed in the initial skeletal muscle-derived stem cell colonies and the skeletal muscle-derived stem cell colonies cultured with a passage number of 3, but is less expressed in the differentiated adult cells. Myostatin, on the other hand, showed an expression pattern opposite to that of follistatin, thus confirming that myostatin is less expressed in the undifferentiated cells.

The initial (passage number 0) skeletal muscle-derived stem cells obtained in Example 9 were cultured at 37° C. under 5% CO₂ for 1 day in the culture medium B supplemented with 0.5 μg/mL or 1.0 μg/mL of myostatin. The resulting skeletal muscle-derived stem cells were analyzed by Western blotting analysis for their expression of p21, Cdk2 and Rb. The results are shown in FIG. 23B. From these results, it was revealed that when the stem cells are cultured in the myostatin-containing culture medium, the expression of the cell cycle inhibitory factor p21 that is a cell growth inhibitory factor is increased, whereas the expression of Cdk2 that is a cell growth promoting factor is decreased. Dephosphorylation of Rb was also recognized.

### Example 11 Investigation of the Influence of Myostatin and Follistatin on Proliferation of the Skeletal Muscle-Derived Stem Cells

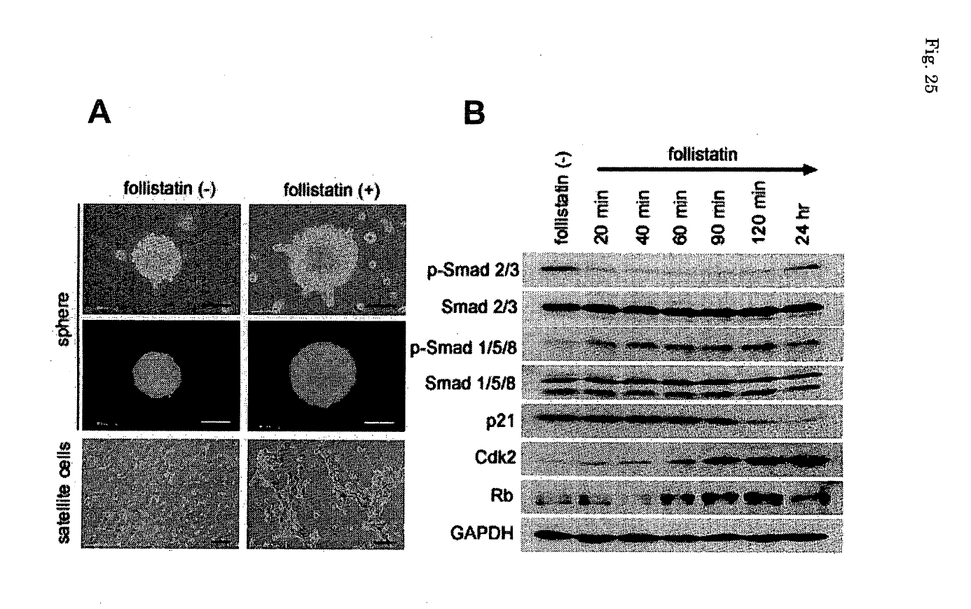

According to the method described in (1) in Example 1, skeletal muscle tissue-derived stem cells were obtained from a myostatin-deficient mouse (mstn−/−) and a GFP-expressing mouse (GFP-Tg), respectively. Then, the myostatin-deficient mouse-derived cells and the GFP-expressing mouse-derived cells were co-cultured at 37° C. under 5% CO₂ for 14 days in the culture medium B. After culture, the results of observation of the shapes of floating stem cell colonies and adherent satellite cells are shown in FIG. 24. Although the myostatin-deficient mouse-derived stem cell colonies are not different in size from the GFP-expressing mouse-derived stem cell colonies, the phenomenon of cell fusion of the myostatin-deficient mouse-derived satellite cells was significantly recognized as compared with the GFP-expressing mouse-derived satellite cells.

The skeletal muscle-derived stem cells obtained in Example 1 were cultured at 37° C. under 5% CO₂ for 14 days in the culture medium A containing 625 ng/mL of follistatin or in the follistatin-free culture medium A. The result indicated that when the skeletal muscle-derived stem cells were cultured in the follistatin-containing culture medium, the amplification rate of the skeletal muscle-derived stem cell colonies was increased and the colony diameter was increased, while the cell-cell fusion of the satellite cells was significantly recognized (see FIG. 25A). Also, the skeletal muscle-derived stem cells obtained in Example 1 were cultured at 37° C. under 5% CO₂ in the culture medium B containing 625 ng/mL of follistatin, and their proteins were extracted with time to analyze expression of p-smad2/3, smad2/3, p-smad1/5/8, smad1/5/8, p21, Cdk2 and Rb by Western blotting analysis. As a result, it was able to be confirmed that follistatin promotes the inactivation of smad2/3 and activation of smad1/5/8 in the skeletal muscle-derived stem cells and simultaneously contributes to a decrease in expression of p21 that is a cell cycle inhibitory factor, to an increase in expression of Cdk2 that is a cell growth promoting factor, and to phosphorylation of Rb, thus promoting proliferation of the stem cell colonies.

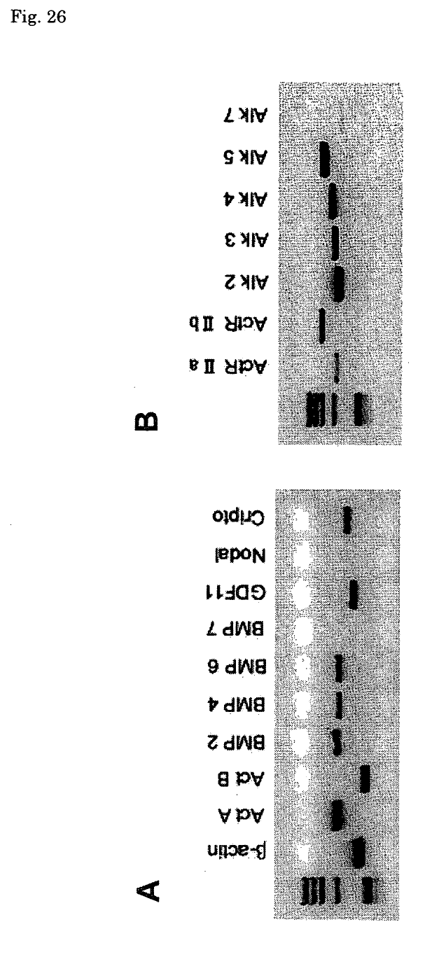

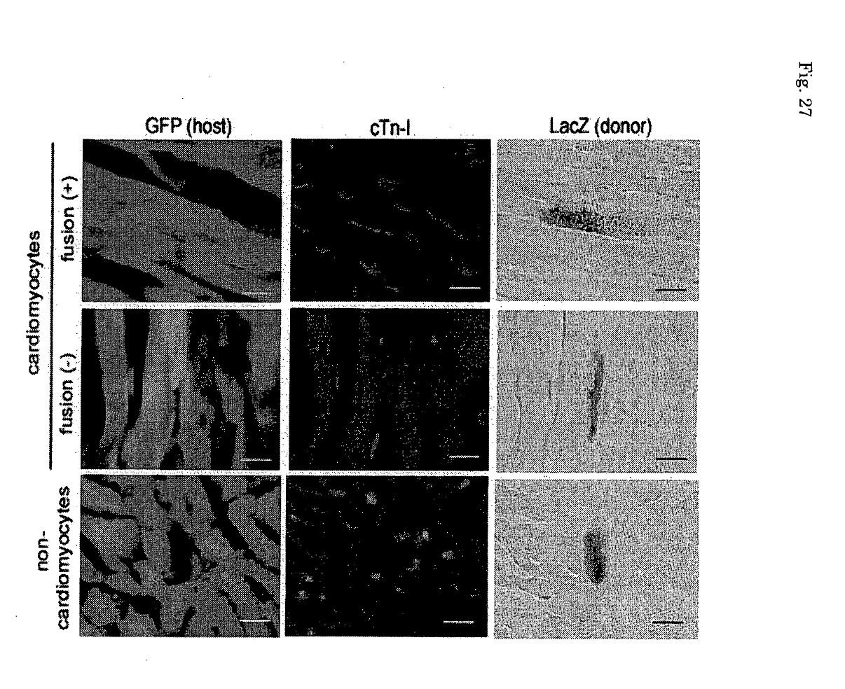

Further, the initial (passage number 0) skeletal muscle-derived stem cells obtained in Example 9 were analyzed by PCR for their expression of Nodal, Activin and GDF11. From these results, it was suggested that the ligands directly controlled by follistatin may be Activin and GDF11 (see FIG. 26A). Expression of activin receptors and ALK2, 3, 4, 5 and 7 in the skeletal muscle-derived stem cells was confirmed by PCR analysis.

### Example 12 Investigation of Differentiated Form of Myocardial Cells Just After Transplantation of Skeletal Muscle-Derived Stem Cells

From 6- to 8-week-old LacZ reporter mice (supplied by Dr. Miyazaki, Medical School, Osaka University), skeletal muscle-derived stem cells were obtained in the same manner as in (1) to (3) in Example 1. The resulting skeletal muscle-derived stem cells (about 1×10⁶ cells) were suspended in 15 μl of PBS(−) (manufactured by GIBCO) to give a skeletal muscle-derived stem cell suspension. Separately, infarcted cardiac muscles were generated in 10- to 12-week-old CAG-EGFP mice having an ability to express GFP (supplied by Dr. Okabe, Medical School, Osaka University) (sometimes referred to hereinafter as GFP-expressing mice). 15 μl of the above skeletal muscle-derived stem cell suspension was transplanted via BD Ultra Fine II Lancet (manufactured by Becton Dickinson) into the infarcted cardiac muscles of the GFP-expressing mice. Twenty-eight days after the stem cells were transplanted, the heart was excised from each mouse. The heart muscle of the excised heart was subjected to cardiac-specific structural protein troponin-I (cTnI) staining (recognized as red color) and LacZ staining (recognized as blue color).

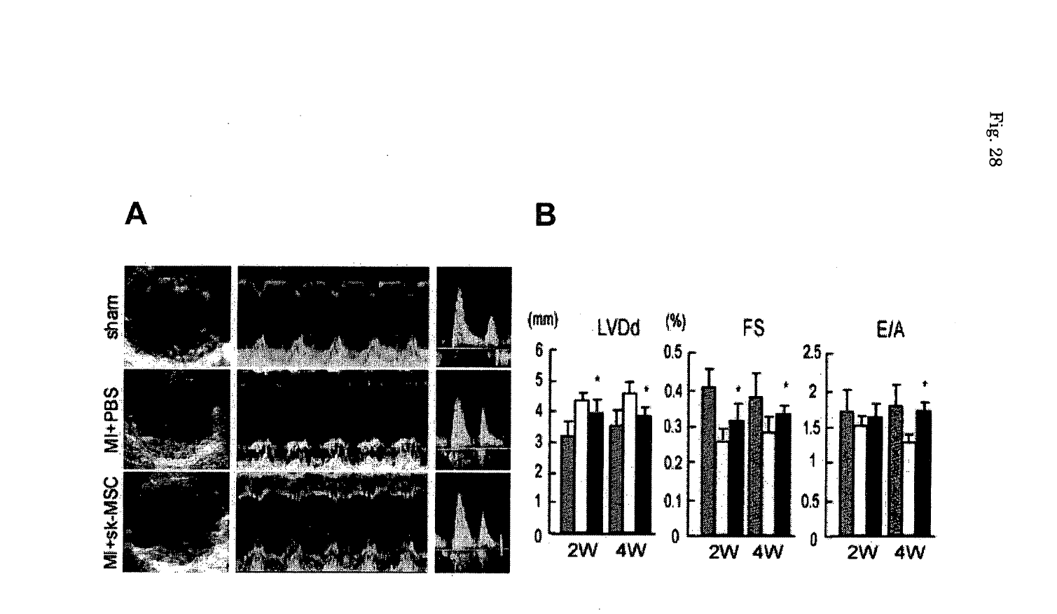

As a result, GFP-positive, LacZ-positive and cTnI-positive myocardial cells were confirmed, and it was thus confirmed that the transplanted, skeletal muscle-derived stem cells were fused with cells of the host heart muscle and differentiated into myocardial cells (see the upper photographs in FIG. 27). The presence of GFP-negative, LacZ-positive and cTnI-positive myocardial cells was also confirmed, and it was thus confirmed that the transplanted, skeletal muscle-derived stem cells were also differentiated positively into myocardial cells without cell-cell fusion (see the middle photographs in FIG. 27). Further, GFP-negative, troponin-I-negative, and LacZ-positive cell were also partially observed, and the presence of cells differentiated into non-myocardial cells was also suggested (see the lower photographs in FIG. 27). When the mice after transplantation were analyzed for their cardiac functions, an improvement in the cardiac functions was recognized, thus confirming the effectiveness of transplantation of the skeletal muscle-derived stem cells.

### Example 13 Analysis of Cardiac Functions in Infarcted Cardiac Muscle Transplanted with Skeletal Muscle-Derived Stem Cells

The skeletal muscle-derived stem cells (about 1×10⁶ cells) obtained in Example 1 were suspended in 15 μl of PBS(−) (manufactured by GIBCO) to give a skeletal muscle-derived stem cell suspension. Separately, infarcted cardiac muscles were generated in 10- to 12-week-old female C57Bl/6J mice (available from Shimizu Laboratory Supplies Co., Ltd.). 15 μl of the above skeletal muscle-derived stem cell suspension was transplanted via BD Ultra Fine II Lancet (manufactured by Becton Dickinson) into the infarcted cardiac muscles of the C57Bl/6J mice. Fourteen days and twenty-eight days after the stem cells were transplanted, their cardiac functions were analyzed by echocardiography. Further, left ventricular end-diastolic diameter, fractional shortening, and left ventricular diastolic performance were measured. For comparison, a group of female C57Bl/6J mice with generated infarcted cardiac muscles to which 15 μl of PBS was administered (MI+PBS group), and a group of C57Bl/6J mice wherein infarcted cardiac muscles were not generated (sham group), were also similarly analyzed for their cardiac functions and cardiac function parameters by echocardiography.

As a result of analysis of cardiac functions by echocardiography, an improvement in wall motion in the ischemic anterior wall region was recognized due to transplantation of the skeletal muscle-derived stem cells (see FIG. 28A). From the measurement results of left ventricular end-diastolic diameter, fractional shortening, and left ventricular diastolic performance, significant improvements in various functions in the mice transplanted with the skeletal muscle-derived stem cells was recognized within 4 weeks after transplantation, as compared with the MI+PBS group mice (see FIG. 28B).

1-23. (canceled)

24. A pluripotent stem cell group composed of pluripotent stem cells derived from a human or mouse skeletal muscle tissue, the pluripotent stem cells being c-met-negative, Pax-7-negative, Myf-5-negative, MyoD-negative, Myogenin-negative, M-cadherin-negative, CD105-positive, CD90-positive, c-kit-negative and CD45-negative, the pluripotent stem cells being CD34-negative in the case of the human-derived stem cells and being CD34-positive in the case of the mouse-derived stem cells, and the pluripotent stem cell group being obtained by proliferation of a single cell.

25. The pluripotent stem cell group according to claim 24, wherein the pluripotent stem cells are Sox-2-positive, Cripto-positive, Nanog-positive, Oct-4-positive, Bmi-1-positive, and Brcp-positive.

26. The pluripotent stem cell group according to claim 24, wherein the pluripotent stem cells have an ability to be differentiated into one or more cells selected from the group consisting of skeletal muscle cell, smooth muscle cell, myocardial cell, blood cell, vascular endothelial cell, fat cell, cartilage cell, osteoblastic cell, and neural cell.

27. The pluripotent stem cell group according to claim 24, wherein the pluripotent stem cells have an ability to be differentiated at least into pulsatile myocardial cells.

28. The pluripotent stem cell group according to claim 24, which is obtained through the following steps:
(i) collecting skeletal muscle tissue from a mammal and enzymatically treating the obtained skeletal muscle tissue to prepare skeletal muscle tissue-derived cell;
(ii) culturing the obtained skeletal muscle tissue-derived cell in a culture medium containing an epidermal growth factor and a fibroblast growth factor;
(iii) selecting and separating a colony that is floating in the culture medium; and
(iv) proliferating cells forming the colony separated in step (iii) above.

29. A method of peparing the pluripotent stem cell group according to claim 24, comprising the following steps:
(i) collecting a skeletal muscle tissue from a mammal and enzymatically treating the obtained skeletal muscle tissue to prepare a skeletal muscle tissue-derived cell;
(ii) culturing the obtained skeletal muscle tissue-derived cell in a culture medium containing an epidermal growth factor and a fibroblast growth factor;
(iii) selecting and separating a colony that is floating in the culture medium; and
(iv) proliferating cells forming the colony separated in step (iii) above.

30. The pluripotent stem cell group according to claim 28, wherein step (iii) comprises selecting a colony formed by proliferation of a single cell from the colonies floating in the culture medium, and then separating the colony in such a manner that only the single colony is present.

31. A method of proliferating pluripotent stem cell derived from a human or mouse skeletal muscle tissue, the pluripotent stem cell being c-met-negative, Pax-7-negative, Myf-5-negative, MyoD-negative, Myogenin-negative, M-cadherin-negative, CD105-positive, CD90-positive, c-kit-negative and CD45-negative, the pluripotent stem cell being CD34-negative in the case of the human-derived stem cell and CD34-positive in the case of the mouse-derived stem cell, the method comprising culturing the pluripotent stem cell in a follistatin-containing culture medium.

32. Use of follistatin for proliferating pluripotent stem cell derived from a human or mouse skeletal muscle tissue, the pluripotent stem cell being c-met-negative, Pax-7-negative, Myf-5-negative, MyoD-negative, Myogenin-negative, M-cadherin-negative, CD105-positive, CD90-positive, c-kit-negative and CD45-negative, the pluripotent stem cell being CD34-negative in the case of the human-derived stem cell and CD34-positive in the case of the mouse-derived stem cell.

33. A therapeutic method for a tissue or organ disease, which comprises transplanting the pluripotent stem cell group of claim 24 or a cell group differentiated from said stem cell group into a patient's tissue or organ.

34. The therapeutic method according to claim 33, wherein the disease to be treated is a cardiac disease.

35. A pharmaceutical composition comprising the pluripotent stem cell group of claim 24.

36. The pharmaceutical composition according to claim 35, which is an agent for regenerating a tissue or a cell.

37. The pharmaceutical composition according to claim 35, which is an agent for treating organ dysfunction.

38. Use of the pluripotent stem cell group of claim 24 in production of a pharmaceutical composition for treatment of a tissue or organ disease.

39. Use according to claim 38, wherein the pharmaceutical composition is an agent for regenerating an organ or a cell.

40. Use according to claim 38, wherein the pharmaceutical composition is an agent for treatment of a cardiac disease.

41. A method of screening for a substance that induces differentiation of the pluripotent stem cell group of claim 24 into various types of cells, which comprises the steps consisting of:
(a) contacting a test substance with the pluripotent stem cell group and culturing the cell group; and
(b) observing whether differentiation induction of the pluripotent stem cell group occurs, and depending on the result, separating the test substance.

42. A method of screening for a substance amplifying the pluripotent stem cell group of claim 24, which comprises the steps consisting of:
(a) contacting a test substance with the pluripotent stem cell group and culturing the cell group; and
(b) observing whether amplification of the pluripotent stem cell group occurs, and depending on the result, separating the test substance.
